(19) United States
(12) Patent Application Publication
(10) Pub. No.: US 2010/0331330 A1
(43) Pub. Date: Dec. 30, 2010

(54) METHODS AND COMPOSITIONS FOR TREATING ISCHEMIC STROKE

(76) Inventors: Gary E. Landerth, Shaker Heights, OH (US); Sophia Sundararajan, Kirtland, OH (US); Colin Combs, Grand Forks, ND (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/438,478

(22) PCT Filed: Aug. 21, 2007
(86) PCT No.: PCT/US07/76381

§ 371 Date: Aug. 18, 2010

Related U.S. Application Data
(60) Provisional application No. 60/839,493, filed on Aug. 23, 2006.

(51) Int. Cl. A61K 31/5377; A61K 31/426; A61K 31/505; A61K 31/47; A61K 31/41; A61K 31/421; A61P 25/00

(52) U.S. Cl. 514/236.8; 514/369; 514/256; 514/311; 514/360; 514/376

(57) ABSTRACT

A method of treating a cerebrovascular accident in a subject includes administering a therapeutically effective amount of at least one PPARγ agonist or a derivative thereof to the subject after onset of ischemia sufficient to a cause the cerebrovascular accident and prior to reperfusion.

### RELATED APPLICATION

This application claims priority from U.S. Provisional Application No. 60/839,493, filed Aug. 23, 2006, the subject matter which is incorporated herein by reference.

### GOVERNMENT FUNDING

This invention was made with government support under Grant No. AG016740 awarded by The National Institute of Health. The government has certain rights in the invention.

### FIELD OF THE INVENTION

The present invention relates to methods and compositions used for treating an acute neurological injury with or without thrombolytic therapy and treating reperfusion injury which may be caused by thrombolytic therapy.

### BACKGROUND

Stoke occurs predominately in the middle and late years of life. Stroke is the third leading cause of death and the leading cause of disability in the U.S. Categories of stroke include ischemia infarction, which is caused by a blockage of an intercerebral artery, and intracranial hemorrhage, which is caused by the rupture of a blood vessel. Symptoms of stroke include an abrupt onset of a focal neurologic deficit, which may remain constant, or may improve or worsen.

Currently the only FDA approved therapy for ischemic stroke is reperfusion mediated by the thrombolytic, tissue plasminogen activator (tPA). While early reperfusion limits ischemic injury and is clearly beneficial, it does allow reperfusion injury to occur. One mechanism implicated in reperfusion injury is a more robust inflammatory response mediated by leukocytes, which become activated within 30 minutes of ischemic onset. Activated leukocytes adhere to adhesion molecules, including selectins, intracellular cell adhesion molecule (ICAM) and vascular adhesion molecule, on the surface of activated endothelium and this accumulation within the microvasculature can lead to capillary plugging, thereby worsening ischemia. Once adherent, leukocytes can transmigrate through the endothelium and into brain parenchyma. Neutrophil infiltrates are detected as early as one hour after reperfusion and are significant six hours after reperfusion. Monocyte infiltration become significant approximately twenty-four hours after reperfusion. In permanent ischemia leukocyte infiltration occurs at later times and is less robust, mostly occurring at the edge of infarction and not in the ischemic core. Activated leukocytes release proteases, lipid derived mediators and reactive oxygen species (ROS) that can further injure already compromised brain. Neutrophil infiltrates are detected as early as one hour after reperfusion and are significant six hours after reperfusion.

### SUMMARY OF THE INVENTION

The present invention relates to a method of treating ischemic stroke in a subject. The stroke can include, for example, an acute ischemic stroke, due to either thrombosis or embolism, or a transient ischemic attack. In the method, a therapeutically effective amount of at least one PPARγ agonist or a derivative thereof is administered to the subject after ischemic onset sufficient to a cause symptoms of stroke and prior to reperfusion. The PPARγ agonist or derivative thereof is administered to the subject in an amount effective to suppress to ICAM expression in the subject. The PPARγ agonist or derivative thereof can also be administered in an amount effective to inhibit or suppress leukocyte infiltration to ischemic tissue of the subject. In another aspect of the invention, the PPARγ agonist or derivative thereof can be administered at an amount effective to mitigate reperfusion related ischemic injury.

The PPARγ agonist or derivative thereof can be administered orally and/or intravenously to the subject. The intravenously administered PPARγ agonist or derivative thereof can be provided in dimethyl sulfoxide solution. The amount of PPARγ agonist or derivative thereof administered to the subject can depend on the specific PPARγ agonist or derivative thereof selected. For example, troglitazone can be administered to a subject at a dose of about 70 mg/kg, pioglitazone can be administered to a subject at a dose of about 1 mg/kg, rosiglitazone can be administered to a subject at a dose of about 0.1 mg/kg.

In one aspect of the invention the PPARγ agonist or a derivative thereof comprises thiazolidinedione or a derivative thereof. In another aspect of the invention, the PPARγ agonist or a derivative thereof comprises at least one compound or a pharmaceutically salt thereof selected from the group consisting of (+)-5[[4-[(3,4-dihydro-6-hydroxy-2,5,7,8-tetramethyl-2H-1-benzopyran-2-yl)methoxy]phenyl]methyl]-2,4-thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4-dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

In another aspect of the invention, a thrombolytic agent can be administered to the subject after administering the PPARγ agonist or derivative thereof. The thrombolytic agent can comprise any agent that is effective in helping to dissolve or break up an occluding thrombus. The thrombolytic agent can be used to reperfuse the occluded tissue.

The present invention also relates to a method of mitigating reperfusion related ischemic injury. In the method, a therapeutically effective amount of at least one PPARγ agonist or a derivative thereof is administered to the subject after ischemic onset sufficient to cause a cerebrovascular accident in a tissue and prior to reperfusion of the tissue. The PPARγ agonist or derivative thereof can be administered to the subject in an amount effective to suppress to ICAM expression in the subject. The PPARγ agonist or derivative thereof can also be administered at an amount effective to inhibit or suppress leukocyte infiltration to ischemic tissue of the subject.

The PPARγ agonist or derivative thereof can be administered prior to the administration of a thrombolytic agent and comprise at least one compound or a pharmaceutically salt thereof selected from the group consisting of (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

### BRIEF DESCRIPTION OF THE DRAWINGS

FIG. 1 illustrates dose response graphs for rosiglitazone and pioglitazone. Rats were injected with either vehicle (DMSO) or various doses of either rosiglitazone or pioglitazone twenty-four hours before and again at the time of 2 hour middle cerebral artery occlusion (MCAO). Vehicle injection resulted in large infarctions encompassing the majority of the MCA distribution. Rosiglitazone at a dose of 0.05 mg/kg did not significantly reduce infarction volume relative to the volume of vehicle injected rats. Rosiglitazone at a dose of 0.1 mg/kg significantly reduced infarction volume, however, that protection was lost when higher doses of 0.35 mg/kg and 1 mg/kg were used (A; student's t-test p=1.5237×10−12; n=16 for vehicle, n=5 for 0.05, 4 for 0.1 mg/kg, 7 for 0.35 mg/kg and 7 for 1 mg/kg). Pioglitazone at a dose of 0.5 mg/kg did not significantly reduce infarction volume relative to the volume of vehicle injected rats. Pioglitazone at a dose of 1.0 and 3.5 mg/kg did significantly reduce infarction volume, however, that protection was lost when a higher dose of 7 mg/kg was used (B; student's t-test; p=9.5922×10−13 for 1 mg/kg and p=7.05571×10−14 for 3.5 mg/kg; n=16 for vehicle, n=8 for 0.5 mg/kg, n=7 for 1 mg/kg, n=8 for 3.5 mg/kg and n=5 for 7 mg/kg).

FIG. 2 illustrates that thiazolidinediones (TZDs) given prior to reperfusion reduce infarction volume. Pioglitazone (1 mg/kg) IP or vehicle (DMSO) was administered to rats IP 24 hours before and again at the time of MCAO. Occlusion was maintained for either 2 hours or permanently.

Twenty-four hours after MCAO animals were sacrificed and infarct volume calculated. Pioglitazone significantly reduced infarction volume in transient ischemia relative to vehicle treated rats. However, during permanent ischemia, no significant protection was evident (A; student's t-test p=0.031; n=7 vehicle injected rats and pioglitazone injected with 2 hour MCAO; n=3 pioglitazone injected with permanent MCAO). The timewindow for pioglitazone's neuroprotection was assessed in 2 hour MCAO model. Pioglitazone 1 mg/kg was administered IP to rats at either 1 hour, 3 hours or 6 hours after onset of 2 hour MCAO. For comparison another set of animals received vehicle treatment 24 hours before and at the time of MCAO. Rats were sacrificed 24 hours after MCAO and infarct volume determined. Infarct volume was significantly reduced when pioglitazone was administered one hour after MCAO onset when compared to vehicle treated rats. There as no significant change in infarct volume when pioglitazone was administered 3 or 6 hours after 2 hour MCAO onset (B; students t-test p=0.006; n=4 pioglitazone injected rats at 1 and 6 hours; n=3 pioglitazone injected rats at 3 hours and n=6 for vehicle injected rats).

FIG. 3 illustrates the delay of reperfusion past the time of TZD administration reduces infarction volume. Rats were subjected to MCAO. Three hours later these rats were injected with either rosiglitazone (0.1 mg/kg; A) or pioglitazone (1 mg/kg; B) dissolved in DMSO. Half the rats were subjected to 2 hour MCAO while the other half underwent 3 hour 15 minute MCAO. For both rosiglitazone and pioglitazone treated rats, those reperfused after 2 hours and treated with TZD one hour later had infarction volumes similar to those previously seen in untreated and vehicle treated rats. However, rats in which reperfusion was delayed until 15 minutes after TZD treatment had significantly reduced infarction volumes relative to rats reperfused at two hours (A, B; student's t-test; p=0.039 for rosiglitazone treated rats; n=7 for rosiglitazone treated rats reperfused at two hours, n=5 for rosiglitazone treated rats reperfused at 3 hours and 15 minutes, p=0.003 for pioglitazone treated rats; n=3 hours for pioglitazone treated rats reperfused at two hours, n=7 for pioglitazone treated rats reperfused at 3 hours and 15 minutes).

FIG. 4 illustrates that myeloperoxidase-IR cells are reduced in animals in which reperfusion is delayed past TZD treatment. Rats were subjected to MCAO. Three hours later these rats were injected with either rosiglitazone (0.1 mg/kg; A) or pioglitazone (1 mg/kg; B) dissolved in DMSO. Half the rats were subjected to 2 hour MCAO while the other half underwent 3 hour 15 minute MCAO. Frozen sections of the rats' brains were incubated with rabbit anti-myeloperoxidase antibodies. Primary antisera were visualized using goat anti-rabbit-Oregon-Green. Myeloperoxidase-IR cells were more abundant in sections of brain from rosiglitazone treated rats reperfused at 2 hours and treated at 3 hours (A) than in sections of brain from rosiglitazone treated rats reperfused at 3.25 hours and treated at 3 hours (B). The number of myeloperoxidase-IR cells was also quantified by counting the number of myeloperoxidase-IR cells per high powered field (360 μm2). The number of immunoreactive cells is significantly reduced when reperfusion is delayed past the time of either rosiglitazone (C) or pioglitazone (D; student's t-test; p=0.007320 for rosiglitazone and p=4.5994×10−10 for pioglitazone; n=for rosiglitazone treatment is 334 HPF from 5 rats for 2 hour MCAO and 378 HPF from 4 rats for 3.25 hour MCAO. For pioglitazone treated rats n=252 HPF from 3 rats for 2 hour MCAO and 640 HPF from 6 rats for 3.25 hour MCAO).

FIG. 5 illustrates that ICAM expression is reduced by TZDs. Frozen sections of brains from rats treated with either vehicle (DMSO), rosiglitazone (0.1 mg/kg) and pioglitazone (1 mg/kg) 24 hours before and again at the time of MCAO and sacrificed 24 hours later. Sections were incubated with mouse anti-ICAM antibody and primary antibody visualized with goat anti-mouse antibody conjugated to Oregon-green. While abundant ICAM-IR is evident is sections from vehicle treated rats (A), less is seen in sections from rosiglitazone (B) and pioglitazone (C) treated rats. In addition, RNA from rats treated with either vehicle, rosiglitazone (0.1 mg/kg), pioglitazone (1 mg/kg) injected 24 hours before and again at the time of 2 hour MCAO and sacrificed twenty-four hours later, was isolated and cDNA transcribed. Real time PCR was performed and showed that there was a significant reduction in ICAM mRNA in both rosiglitazone and pioglitazone treated rats compared with vehicle treated rats (student's t-test; p=0.016 for rosiglitazone; n=3 for both vehicle and rosiglitazone treatment; p=; n=4 for vehicle and 5 for pioglitazone treatment).

FIG. 6 illustrates that PPARγ mRNA is increased in ischemic brains relative to brains from sham-operated rats at 24 hours. mRNA was isolated from brains subjected to either sham operation or MCAO twenty-four hours prior. Real time PCR was performed on a BioRad iCycler and PPARγ cycle thresholds were normalized against the values obtained for β-actin. p<0.05; n=3 sham and 5 for MCAO. Error bars represents standard error of the mean.

FIG. 7 illustrates that PPARγ-IR neuronal cell bodies are not seen in sham-operated rats, but are present following MCAO. Frozen sections of rat brain were incubated with a rabbit anti-PPARγ antisera and a mouse antibody directed against the neuronal marker, NeuN. Primary antibodies were visualized using a goat anti-rabbit biotinylated antibody and goat anti-mouse-Oregon green. Strepavidin-Cy-3 was used to detect the biotinylated antibody. A representative section is shown from sham operated animals labeled with anti-PPARγ antisera (A). The same high powered field labeled with NeuN antibodies (B) and the overlay of the two fields (C). A representative section from a rat exposed to two hour MCAO and labeled with anti-PPARγ antisera (D). The same high powered field labeled with NeuN antibodies (E). The overlay of the two fields is shown in (F). The scale bar represents 25 μm.

FIG. 8 illustrates that PPARγ-IR increases during the first twenty-four hours after MCAO and remains elevated for two weeks. Frozen sections of brain were incubated with rabbit anti-PPARγ antiserum. Primary antisera were visualized using goat anti-rabbit biotin and strepavidin conjugated to Cy-3. Representative sections from animals sacrificed four hours (A), 12 hours (B), 24 hours (C) and two days (D) after MCAO. Arrows indicate PPARγ-IR cell bodies. The scale bar represents 50 μm. Quantification was obtained by counting PPARγ-IR cell bodies from high powered fields (HPFs; 360 μm2) covering entire sections at the different time points. Cell counts which were significantly different from sham operated animals are marked with an asterisk (p<0.05; sham n=3 animals, 187 HPF; 4 hour n=4 animals, 241 HPF; 6 hour n=5 animals, 295 HPF; 12 hours n=4 animals, 280 HPF; 24 hours n=5 animals, 349 HPF; 48 hours n=3 animals, 190 HPF; 7 days n=4 animals, 225 HPF; 14 days n=3 animals, 162 HPF; 28 days n=3 animals, 110 HPF). Error bars represent standard error of the mean in one direction.

FIG. 9 illustrates that PPARγ-IR is located primarily in areas exposed to less severe ischemia twenty-four hours after MCAO. Rats underwent MCAO and were euthanized 24 hour later. Frozen sections of their brains were incubated with rabbit anti-PPARγ antiserum. Primary antisera were visualized using goat anti-rabbit biotin and strepavidin conjugated to Cy-3. Representative sections from the frontal lobe (anterior cerebral artery-middle cerebral artery watershed; A), the frontoparietal cortex within the infarction (B), the temporal lobe also within the infarction (C) and the striatum, which represents the ischemic core (D) are shown. Scale bar represents 50 μm.

FIG. 10 illustrates that DNA binding of PPARγ is reduced by MCAO, but increased by PPARγ agonist treatment. Rats were treated with either DMSO alone or rosiglitazone 0.1 mg/kg dissolved in DMSO eight hours twenty-four hours before and again at the time of two hour MCAO. Tissue from the brains was collected eight hours later and nuclear extract incubating with 32P-labeled double stranded oligonucleotide containing the PPAR responsive element sequences in the rat acyl CoA oxidase promoter. Competition assays using 100 fold radioinert competitor oligonucleotides included in the reaction mixture and interference assays with antibody to PPARγ were used to determine specificity of PPARγ-PPRE binding. Nucleoproteinoligonucleotide complexes were resolved electrophoretically on a 7% non-denaturing polyacrylamide gel which was autoradiographed and optical density was assessed using Kodak Analysis (EDAS) 290 system. Samples from the ipsilateral (ischemic; A) and contralateral (non-ischemic; B) hemisphere are shown (V=vehicle treated; R=rosiglitazone treated). Samples from both hemispheres are also shown (C). Densitometry of these blots show that binding is significantly reduced in the ipsilateral hemisphere relative to the contralateral hemisphere and that treatment with rosiglitazone significantly increase PPARγ-PPRE binding in both the ipsilateral and the contralateral hemispheres (D).

FIG. 11 illustrates that Lipoprotein lipase expression is increased following PPARγ ligand treatment; but not in the absence of ligand. Animals were treated with either DMSO alone, pioglitazone 1.0 mg/kg or rosiglitazone 0.1 mg/kg dissolved in DMSO twenty-four hours before and again at the time of two hour MCAO. Tissue from the ipsilateral hemisphere of the brains was collected twenty-four hours later and mRNA isolated from them. LPL mRNA levels were measured using real time PCR and cycle thresholds were normalized against the values obtained for β-actin. mRNA from rats exposed to a sham operation is compared to mRNA from rats exposed to two hours MCAC (A). In addition LPL mRNA is also compared in vehicle, rosiglitazone and pioglitazone treated rats (B; C). Error bar represents standard error of the mean. Statistically significant differences (student's t-test; p<0.05) are marked with an asterisk.

FIG. 12 illustrates that treatment with the PPARγ antagonist T0070907 increases infarction size both in the presence and absence of PPARγ ligand. Rats were treated with either DMSO, rosiglitazone (0.1 mg/kg) dissolved in DMSO or rosiglitazone (0.1 mg/kg) plus T0070907 (1.5 mg/kg) dissolved in DMSO twenty-four hours before and again at the time of two hour MCAO. Animals were allowed to survive for twenty-four hours and infarction volumes were then calculated. Infarction volumes were compared in animals treated with vehicle, rosiglitazone, or rosiglitazone plus T0070907 (A; student's t-test; p<0.05). A separate set of animals was treated with either DMSO or T0070907 twenty-four hours before and again at the time of 90 minute MCAO. Animals were allowed to survive for twenty-four hours and infarction volumes were then calculated. Infarction volumes between vehicle treated and T0070907 treated rats were also compared (B; student's t-test; p<0.05). Values significantly different from vehicle treated rats are marked with an asterisk. Error bars represent standard error of the mean in one direction only. Mean absolute volumes for these infarctions are as follows: A (2 hr MCAO): vehicle 166 mm2, rosiglitazone 50 mm2, rosiglitazone+antagonist 188 mm2 B (90 minute MCAO): vehicle 89 mm2, T0070907 176 mm2. In panel A one animal in the vehicle group died, no animals in either the rosiglitazone or rosiglitazone plus antagonist group died. In panel B one animal in each group died. Data from animals which died are not included in the analysis.

### DETAILED DESCRIPTION

As used herein, the term “therapeutically effective amount” refers to that amount of a composition that results in amelioration of symptoms or a prolongation of survival in a patient. A therapeutically relevant effect relieves to some extent one or more symptoms of a disease or condition or returns to normal either partially or completely one or more physiological or biochemical parameters associated with or causative of the disease or condition.

As used herein, the term “PPARγ agonist” refers to a compound or composition, which when combined with PPARγ, directly or indirectly stimulates or increases an in vivo or in vitro reaction typical for the receptor (e.g., transcriptional regulation activity). The increased reaction can be measured by any of a variety of assays known to those skilled in the art. An example of a PPARγ agonist is a thiazolidinedione compound, such as troglitazone, rosiglitazone, pioglitazone, ciglitazone, WAY-120,744, englitazone, AD 5075, darglitazone, and congeners, analogs, derivatives, and pharmaceutically acceptable salts thereof.

As used herein, the terms “host” and “subject” refer to any animal, including, but not limited to, humans and non-human animals (e.g., rodents, arthropods, insects, fish (e.g., zebrafish), non-human primates, ovines, bovines, ruminants, lagomorphs, porcines, caprines, equines, canines, felines, ayes, etc.), which is to be the recipient of a particular treatment. Typically, the terms “host,” “patient,” and “subject” are used interchangeably herein in reference to a human subject.

The term “biologically active,” as used herein, refers to a protein or other biologically active molecules (e.g., catalytic RNA) having structural, regulatory, or biochemical functions of a naturally occurring molecule.

The term “agonist,” as used herein, refers to a molecule which, when interacting with a biologically active molecule, causes a change (e.g., enhancement) in the biologically active molecule, which modulates the activity of the biologically active molecule. Agonists include, but are not limited to proteins, nucleic acids, carbohydrates, lipids or any other molecules which bind or interact with biologically active molecules. For example, agonists can alter the activity of gene transcription by interacting with RNA polymerase directly or through a transcription factor or signal transduction pathway.

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

As used herein, the term “in vitro” refers to an artificial environment and to processes or reactions that occur within an artificial environment. In vitro environments consist of, but are not limited to, test tubes and cell culture. The term “in vivo” refers to the natural environment (e.g., an animal or a cell) and to processes or reaction that occur within a natural environment.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms. Treatment, prevention and ameliorating a condition, as used herein, can include, for example decreasing the infarct volume of tissue associated with a stroke.

The compositions and methods of the present invention are based on the use of PPARγ agonists to suppress, inhibit, modulate, or mitigate the infarct volume (or size) following a stroke. It was found that PPARγ agonists, such as a thiazolidinedione (e.g., rosiglitazone and pioglitazone), can be administered to a subject up to about three hours after acute ischemic stroke onset and before the blocked vessel is re-opened to prevent, suppress, or reduce reperfusion related ischemic injury and to improve neurological function.

Although it is not necessary to understand the mechanisms in order to practice the present invention, and it is not intended that the present invention be so limited, it is shown by the present invention that in order for PPARγ agonists to be effective in the treatment of acute ischemic stroke, they must be administered before thrombolysis.

PPARγ agonists reduce the expression of adhesion molecules and other genes required for neutrophils to enter the brain. The sooner this down regulation occurs relative to the time of reperfusion, the more effective it will be at limiting infiltration. For example, it was found that PPARγ agonists reduce the levels of ICAM mRNA and protein. ICAM plays an important role in leukocyte infiltration from the vasculature following ischemia and reduced ICAM may be the mechanism by which decreased infiltration of systemic inflammatory cells is achieved. Once activated, leukocytes produce a variety of toxic products which exacerbate injury and enlarge infarction size.

One aspect of the present invention relates to method of treating an acute ischemic stroke in a subject by administering a therapeutically effective amount of compounds that include PPARγ agonists or therapeutically effective derivatives thereof to the subject following onset of acute ischemic stroke and prior to reperfusion. It is desirable that the time of administration of the PPARγ agonists be as soon as possible after ischemic onset and within, for example, about three hours to minimize ischemic injury in the subject.

In an aspect of the invention the PPARγ agonists can include, for example, prostaglandin J2 (PGJ2) and analogs thereof (e.g., A2-prostaglandin J2 and 15-deoxy-2 4-prostaglandin J2), members of the prostaglandin D2 family of compounds, docosahexaenoic acid (DHA), and thiazolidinediones (e.g., ciglitazone, troglitazone, pioglitazone, and rosiglitazone).

In addition, such agents include, but are not limited to, L-tyrosine-based compounds, farglitazar, GW7845, indole-derived compounds, indole 5-carboxylic acid derivatives and 2,3-disubstituted indole 5-phenylacetic acid derivatives. It is significant that most of the PPARγ agonists exhibit substantial bioavailability following oral administration and have little or no toxicity associated with their use (See e.g., Saltiel and Olefsky, Diabetes 45:1661 (1996); Wang et al, Br. J. Pharmacol. 122:1405 (1997); and Oakes et al, Metabolism 46:935 (1997)). It will be appreciated that the present invention is not limited to above-identified PPARγ agonists and that other identified PPARγ agonists can also be used.

PPARγ agonists that can be used for practicing the present invention, and methods of making these compounds are disclosed in WO 91/07107; WO 92/02520; WO 94/01433; WO 89/08651; WO 96/33724; WO 97/31907; U.S. Pat. Nos. 4,287,200; 4,340,605; 4,438,141; 4,444,779; 4,461,902; 4,572,912; 4,687,777; 4,703,052; 4,725,610; 4,873,255; 4,897,393; 4,897,405; 4,918,091; 4,948,900; 5,002,953; 5,061,717; 5,120,754; 5,132,317; 5,194,443; 5,223,522; 5,232,925; 5,260,445; 5,814,647; 5,902,726; 5,994,554; 6,294,580; 6,306,854; 6,498,174; 6,506,781; 6,541,492; 6,552,055; 6,579,893; 6,586,455, 6,660,716, 6,673,823; 6,680,387; 6,768,008; 6,787,551; 6,849,741; 6,878,749; 6,958,355; 6,960,604; 7,022,722 and U.S. Applications 20030130306, 20030134885, 20030109579, 20030109560, 20030088103, 20030087902, 20030096846, 20030092697, 20030087935, 20030082631, 2003007g288, 20030073862, 20030055265, 20030045553, 1 20020169192, 20020165282, 20020160997, 20020128260, 20020103188, 20020082292, 20030092736, 20030069275, 20020151569, and 20030064935.

The disclosures of these publications are incorporated herein by reference in their entireties, especially with respect to the PPARγ agonists disclosed therein, which may be employed in the methods described herein.

As PPARγ agonist having the aforementioned effects, the compounds of the following formulas are useful in treating individuals. Accordingly, in some embodiments of the present invention, the therapeutic agents comprise compounds of

### Formula I:

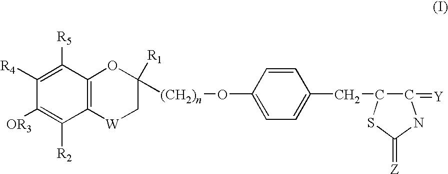

wherein R₁ and R₂ are the same or different, and each represents a hydrogen atom or a C₁-C₅ alkyl group; R₃ represents a hydrogen atom, a C₁-C₆ aliphatic acyl group, an alicyclic acyl group, an aromatic acyl group, a heterocyclic acyl group, an araliphatic acyl group, a (C₁-C₆ alkoxy)carbonyl group, or an aralkyloxycarbonyl group; R₄ and R₅ are the same or different, and each represents a hydrogen atom, a C₁-C₅ alkyl group or a C₁-C₅ alkoxy group, or R₄ and R₅ together represent a C₁-C₅ alkylenedioxy group; n is 1, 2, or 3; W represents the CH₂, CO, or CHOR₆ group (in which R₆ represents any one of the atoms or groups defined for R₃ and may be the same as or different, from R₃); and Y and Z are the same or different and each represents an oxygen atom or an imino (—NH) group; and pharmaceutically acceptable salts thereof.

In some embodiments of the present invention, the PPARγ agonist comprise compounds of Formula II:

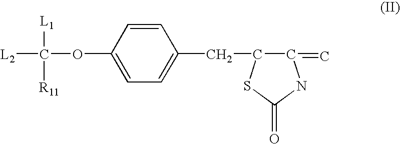

wherein R₁₁ is a substituted or unsubstituted alkyl, alkoxy, cycloalkyl, phenylalkyl, phenyl, aromatic acyl group, a 5- or 6 membered heterocyclic group including 1 or 2 heteroatoms selected from the group consisting of nitrogen, oxygen, and sulfur, or a group of the formula indicated in:

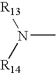

wherein R₁₃ and R₁₄ are the same or different and each is a lower alkyl (alternately, R₁₃ and R₁₄ are combined to each other either directly or as interrupted by a heteroatom comprising nitrogen, oxygen, and sulfur to form a 5- or 6-membered ring); and wherein L¹ and L² are the same or different and each is hydrogen or lower alkyl or L¹ and L² are combined to form an alkylene group; or a pharmaceutically acceptable salt thereof.

In some aspects of the present invention, the therapeutic agents comprise compounds of Formula III:

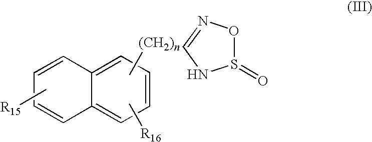

wherein R₁₅ and R₁₆ are independently hydrogen, lower alkyl containing 1 to 6 carbon atoms, alkoxy containing 1 to 6 carbon atoms, halogen, ethyl, nitrite, methylthio, trifluoromethyl, vinyl, nitro, or halogen substituted benzyloxy; n is 0 to 4; or a pharmaceutically acceptable salt thereof.

In some aspects of the present invention, the PPARγ agonist comprise compounds of Formula IV:

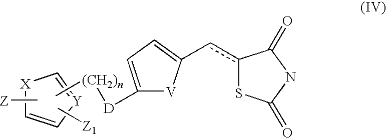

wherein the dotted line represents a bond or no bond; V is HCH—, —NCH—, —CH═N—, or S; D is CH₂, CHOH, CO, C═NOR₁₇, or CH═CH; X is S, SO, NR₁₈, —CH═N, or —N═CH; Y is CH or N; Z is hydrogen, (C₁-C₇)alkyl, (C₁-C₇)cycloalkyl, phenyl, naphthyl, pyridyl, furyl, thienyl, or phenyl mono- or di-substituted with the same or different groups which are (C₁-C₃)alkyl, trifluoromethyl, (C₁-C₃)alkoxy, fluoro, chloro, or bromo; Z₁ is hydrogen or (C₁-C₃)alkyl; R₁₇ and R₁₈ are each independently hydrogen or methyl; and n is 1, 2, or 3; the pharmaceutically acceptable cationic salts thereof; and the pharmaceutically acceptable acid addition salts thereof when the compound contains a basic nitrogen.

In some embodiments of the present invention, the PPARγ agonist comprise compounds of Formula V:

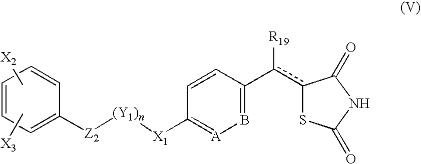

wherein the dotted line represents a bond or no bond; A and B are each independently CH or N. with the proviso that when A or B is N. the other is CH; X is S, SO, SO₂, CH₂, CHOH, or CO; n is 0 or 1; Y₁ is CHR₂₀ or R₂₁, with the proviso that when n is 1 and Yᵢ is NR₂₁, X₁ is SO₂ or CO; Z₂ is CHR₂₂, CH₂CH₂, cyclic C₂H₂O, CH═CH, OCH₂, SCH₂, SOCH₂, or SO₂CH₂; R₁₉, R₂₀, R₂₁, and R₂₂ are each independently hydrogen or methyl; and X₂ and X₃ are each independently hydrogen, methyl, trifluoromethyl, phenyl, benzyl, hydroxy, methoxy, phenoxy, benzyloxy, bromo, chloro, or fluoro; a pharmaceutically acceptable cationic salt thereof; or a pharmaceutically acceptable acid addition salt thereof when A or B is N.

In some embodiments of the present invention, the PPARγ agonist comprise compounds of Formula VI:

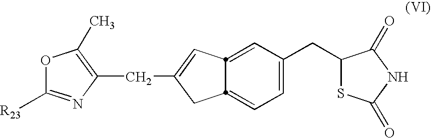

or a pharmaceutically acceptable salt thereof, wherein R₂₃ is alkyl of 1 to 6 carbon atoms, cycloalkyl of 3 to 7 carbon atoms, phenyl or mono- or all-substituted phenyl wherein said substituents are independently alkyl of 1 to 6 carbon atoms, alkoxy of 1 to 3 carbon atoms, halogen, or trifluoromethyl.

In some embodiments of the present invention, the PPARγ agonist comprise compounds of Formula VII:

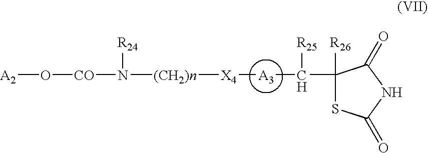

or a tautomeric form thereof and/or a pharmaceutically acceptable salt thereof, and/or a pharmaceutically acceptable solvate thereof, wherein: A₂ represents an alkyl group, a substituted or unsubstituted aryl group, or an aralkyl group wherein the alkylene or the aryl moiety may be substituted or unsubstituted; A³ represents a benzene ring having in total up to 3 optional substituents; R₂₄ represents a hydrogen atom, an alkyl group, an acyl group, an aralkyl group wherein the alkcyl or the aryl moiety may be substituted or unsubstituted, or a substituted or unsubstituted aryl group; or A₂ together with R₂₄ represents substituted or unsubstituted C₂₋₃ polymethylene group, optional substituents for the polymethylene group being selected from alkyl or aryl or adjacent substituents together with the methylene carbon atoms to which they are attached form a substituted or unsubstituted phenylene group; R₂₅ and R₂₆ each represent hydrogen, or R₂₅ and R₂₆ together represent a bond; X₄ represents O or S; and n represents an integer in the range from 2 to 6.

In some embodiments of the present invention, the PPARγ agonists comprise compounds of Formula VIII:

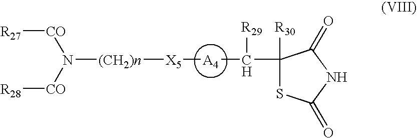

or a tautomeric form thereof and/or a pharmaceutically acceptable salt thereof, and/or a pharmaceutically acceptable solvate thereof, wherein: R₂₇ and R₂₈ each independently represent an alkyl group, a substituted or unsubstituted aryl group, or an aralkyl group being substituted or unsubstituted in the aryl or alkyl moiety; or R₂₇ together with R₂₈ represents a linking group, the linking group consisting or an optionally substituted methylene group or an O or S atom, optional substituents for the methylene groups including alkyl, aryl, or aralkyl, or substituents of adjacent methylene groups together with the carbon atoms to which they are attached form a substituted or unsubstituted phenylene group; R₂₉ and R₃₀ each represent hydrogen, or R₂₉ and R₃₀ together represent a bond; A₄ represents a benzene ring having in total up to 3 optional substituents; X₅ represents O or S; and n represents an integer in the range of 2 to 6.

In some embodiments of the present invention, the PPARγ agonist comprise compounds of Formula IX:

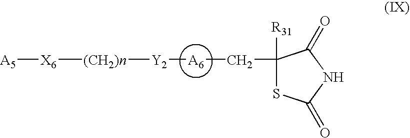

or a tautomeric form thereof and/or a pharmaceutically acceptable salt thereof, and/or a pharmaceutically acceptable solvate thereof, wherein: A₅ represents a substituted or unsubstituted aromatic heterocyclyl group; A₆ represents a benzene ring having in total up to 5 substituents; X₆ represents O, S, or NR₃₂ wherein R₃₂ represents a hydrogen atom, an alkyl group, an acyl group, an aralkyl group, wherein the aryl moiety may be substituted or unsubstituted, or a substituted or unsubstituted aryl group; Y₂ represents O or S; R₃₁ represents an alkyl, aralkyl, or aryl group; and n represents an integer in the range from 2 to 6. Aromatic heterocyclyl groups include substituted or unsubstituted, single or fused ring aromatic heterocyclyl groups comprising up to 4 hetero atoms in each ring selected from oxygen, sulfur, or nitrogen. Aromatic heterocyclyl groups include substituted or unsubstituted single ring aromatic heterocyclyl groups having 4 to 7 ring atoms, preferably 5 or 6 ring atoms.

In particular, the aromatic heterocyclyl group comprises 1, 2, or 3 heteroatoms, especially 1 or 2, selected from oxygen, sulfur, or nitrogen. Values for A₅ when it represents a 5-membered aromatic heterocyclyl group include thiazolyl and oxazoyl, especially oxazoyl. Values for A₆ when it represents a 6 membered aromatic heterocyclyl group include pyridyl or pyrimidinyl. R₃₁ represents an alkyl group, in particular a C-6 allyl group (e.g., a methyl group).

A⁵ can represent a moiety of formula (a), (b), or (c), under Formula IX:

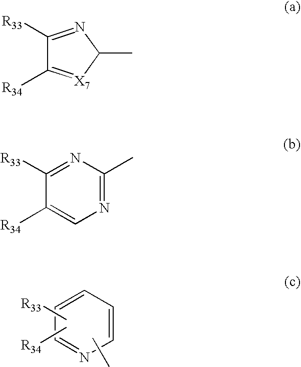

wherein, R₃₃ and R₃₄ each independently represents a hydrogen atom, an alkyl group, or a substituted or unsubstituted aryl group or when R₃₃ and R₃₄ are each attached to adjacent carbon atoms, then R₃₃ and R₃₄ together with the carbon atoms to which they are attached forth a benzene ring wherein each carbon atom represented by R₃₃ and R₃₄ together may be substituted or unsubstituted; and in the moiety of Formula (a), X₇ represents oxygen or sulphur.

In one embodiment of the present invention, R₃₃ and R₃₄ together present a moiety of Formula (d) in FIG. 8, under Formula IX:

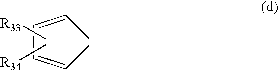

wherein R₃₅ and R₃₆ each independently represent hydrogen, halogen, substituted or unsubstituted alkyl, or alkoxy.

In some embodiments of the present invention, the PPARγ agonist comprise compounds of Formula X:

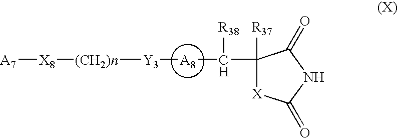

or a tautomeric form thereof and/or a pharmaceutically acceptable salt thereof, and/or a pharmaceutically acceptable solvate thereof, wherein: A₇ represents a substituted or unsubstituted aryl group; A₈ represents a benzene ring having in total up to 5 substituents; X₈ represents O, S, or NR₉, wherein R₃₉ represents a hydrogen atom, an alkyl group, an acyl group, an aralkyl group, wherein the aryl moiety may be substituted or unsubstituted, or a substituted or unsubstituted aryl group; Y₃ represents O or S; R₃₇ represents hydrogen; R₃₈ represents hydrogen or an alkyl, aralkyl, or aryl group or R₃₇ together with R₃₈ represents a bond; and n represents an integer in the range from 2 to 6.

In some embodiments of the present invention, the PPARγ agonist comprise compounds of Formula XI:

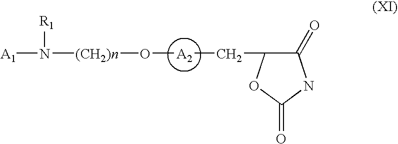

or a tautomeric form thereof and/or a pharmaceutically acceptable salt thereof, and/or a pharmaceutically acceptable solvate thereof, wherein: A₁ represents a substituted or unsubstituted aromatic heterocyclyl group; R₁ represents a hydrogen atom, an alkyl group, an acyl group, an aralkyl group, wherein the aryl moiety may be substituted or unsubstituted, or a substituted or unsubstituted aryl group; A₂ represents a benzene ring having in total up to 5 substituents; and n represents an integer in the range of from to 6. Suitable aromatic heterocyclyl groups include substituted or unsubstituted, single or fused ring aromatic heterocyclyl groups comprising up to 4 hetero atoms in each ring selected from oxygen, sulfur, or nitrogen. Favored aromatic heterocyclyl groups include substituted or unsubstituted single ring aromatic heterocyclyl groups having 4 to 7 ring atoms, preferably 5 or 6 ring atoms. In particular, the aromatic heterocyclyl group comprises 1, 2, or 3 heteroatoms, especially 1 or 2, selected from oxygen, sulfur, or nitrogen. Values for A₁ when it represents a 5-membered aromatic heterocyclyl group can include thiazolyl and oxazolyl, especially oxazoyl. Values for A₁ when it represents a 6-membered aromatic heterocyclyl group can include pyridyl or pyrimidinyl.

In some embodiments of the present invention, the PPARγ agonists comprise a compound of Formulas XII and XIII:

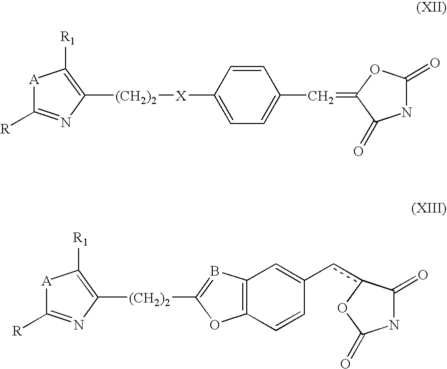

or pharmaceutically acceptable salts thereof wherein the dotted line represents a bond or no bond; R is cycloalkyl of three to seven carbon atoms, naphthyl, thienyl, furyl, phenyl, or substituted phenyl wherein the substituent is alkyl of one to three carbon atoms, alkoxy of one to three carbon atoms, trifluoromethyl, chloro, fluoro, or bis(trifluoromethyl); R₁ is an alkyl of one to three carbon atoms; X is O or C═O; A is O or S; and B is N or CH.

Some embodiments of the present invention include the use of the compounds of Formulas I through XIII are referred to as thiazolidine derivatives. Where appropriate, the specific names of thiazolidine derivatives may be used including: troglitazone, ciglitazone, pioglitazone, and rosiglitazone.

In certain embodiments, the therapeutic agent comprises an activator of PPARγ as described in U.S. Pat. No. 5,994,554, e.g., having a structure selected from the group consisting of formulas (XIV)-(XXVI):

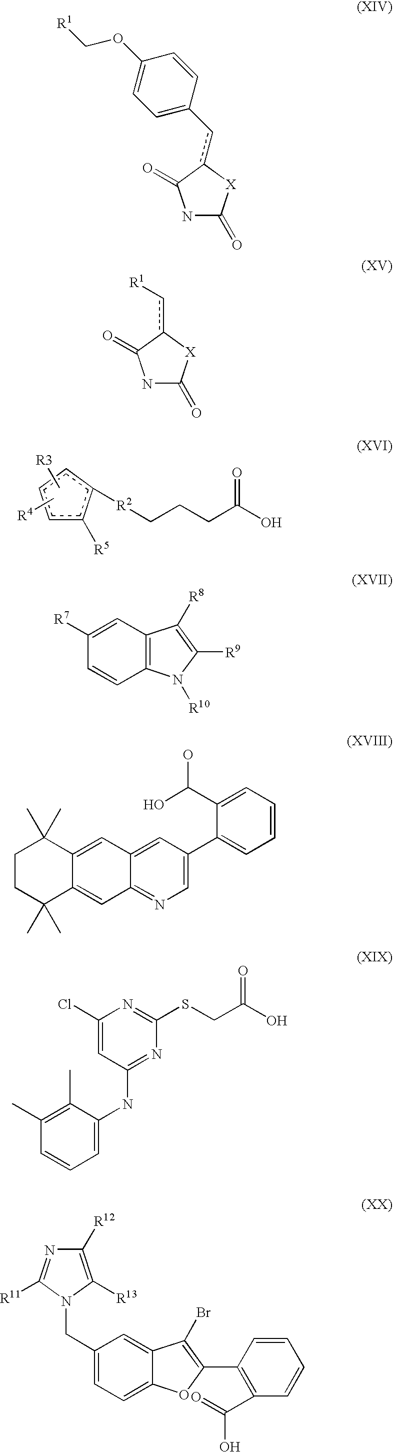

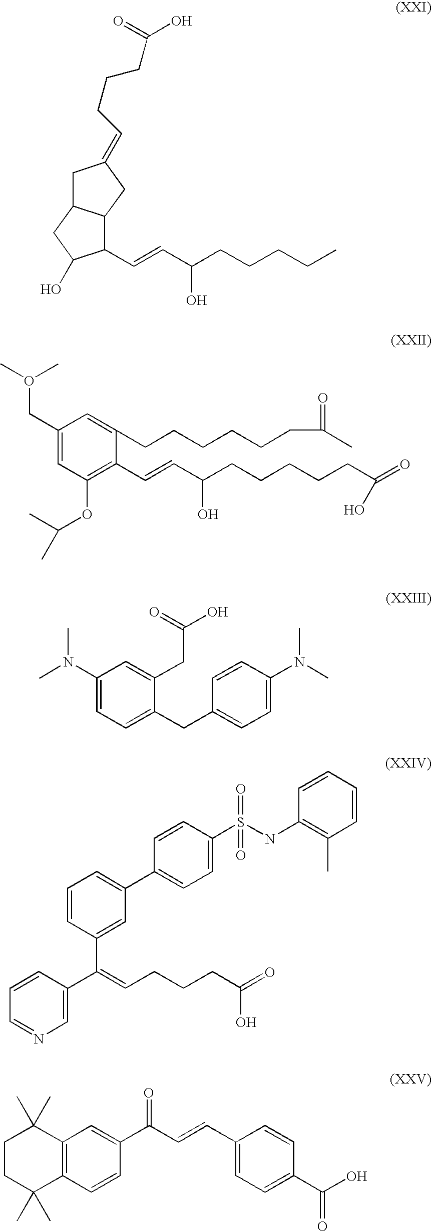

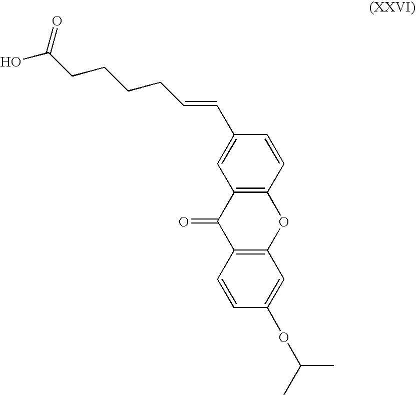

wherein: R¹ is selected from the group consisting of hydrogen, C₁₋₈ alkyl, aminoC₁₋₈, alkyl, C₁₋₈alkylamino C₁₋₈ alkyl, heteroarylamino C₁₋₆ alkyl, (heteroaryl)(C₁₋₈alkyl)aminoC₁₋₆ alkyl, (C₁₋₈ cycloalkyl) C₁₋₈ alkyl, C₁₋₈ alkylheteroaryl C₁₋₈ alkyl, 9- or 10-membered heterobicycle, which is partially aromatic or substituted 9- or 10-membered heterobicycle, which is partially aromatic; X is selected from the group consisting of S, NH, or O; R² is selected from the group consisting of hydrogen, C₁₋₈allyl or C₁₋₈alkenyl; R³ and R⁴ are independently selected from the group consisting of hydrogen, hydroxy, oxo C₁₋₈alkyl, C₁₋₈alkoxy or amino; R⁵ is selected from the group consisting of hydrogen, C₁₋₈alkyl, C₁₋₈alkenyl, (carbonyl)alkenyl, (hydroxy)alkenyl, phenyl, C₁₋₈alkylR⁶, (hydroxy) C₁₋₈alkylR⁶, C₁₋₈alkyl C₁₋₈cycloallylR⁶, (hydroxy) C₁₋C₁₋₈cycloallylR⁶ or C₁₋₈cycloallylthioR⁶; R⁶ is selected from the group consisting of phenyl or phenyl substituted with hydroxy, C₁₋₈alkyl or C₁₋₈alkoxy substituents; R⁷ is selected from the group consisting of hydrogen, hydroxy, carboxy or carboxy C₁₋₈alkyl; R⁸ is selected from the group consisting of hydrogen, C₁₋₈alkyl, phenyl, phenyl C₁₋₈alkyl, phenyl mono- or all-substituted with halo, hydroxy, and/or C₁₋₈alkoxy (e.g., methoxy) substituents or phenyl C₁₋₈alkyl wherein the phenyl is mono- or disubstituted with halo, hydroxy, and/or C₁₋₈alkoxy (e.g., methoxy) substituents; R⁹ is selected from the group consisting of hydrogen, C₁₋₈alkyl, carboxy C₁₋₈alkenyl mono- or disubstituted with hydroxy, and/or C₁₋₈alkoxy (e.g., methoxy), phenyl or phenyl mono- or disubstituted with halo, hydroxy, and/or C₁₋₈alkoxy (e.g., methoxy) R¹⁰ is hydrogen or C₁₋₈alkyl, R¹¹ is selected from the group consisting of hydrogen, C₁₋₈alkyl or cycloC₁₋₈alkyl C₁₋₈alkyl; R¹² is selected from the group consisting of hydrogen, halo or fluorinated C₁₋₈alkyl; R¹³ is selected from the group consisting of hydrogen, C₁₋₈alkoxycarbonyl or C₁₋₈alkoxycarbonyl C₁₋₈alkylaminocarbonyl; a dashed line (---) is none or one double bond between two of the carbon atoms; fluorinated alkyl can be an alkyl wherein one or more of the hydrogen atoms is replaced by a fluorine atom; heteroaryl can be 5, 6 or 7 membered aromatic ring optionally interrupted by 1, 2, 3 or 4 N, S, or O heteroatoms, with the proviso that any two O or S atoms are not bonded to each other; substituted heteroaryl can be a 9- or 10-membered heterobicycle mono-, di-, or trisubstituted independently with hydroxy, oxo, C₁₋₆ alkyl, C₁₋₆ alkoxy or 9- or 10-membered heterobicycle, which is partially aromatic in more detail is a heterobicycle interrupted by 1, 2, 3, or 4 N heteroatoms; substituted 9- or 10-membered heterobicycle, which is partially aromatic in more detail is a 9- or 10-membered heterobicycle mono-, di-, tri- or tetrasubstituted independently with hydroxy, oxo, C₁₋₈ alkyl, C₁₋₈ alkoxy, phenyl, phenyl C₁₋₈ alkyl; or a pharmaceutically acceptable acid-addition or base-addition salt thereof.

In yet other embodiments, the PPARγ agonist comprises a compound as disclosed in U.S. Pat. No. 6,306,854, e.g., a compound having a structure of Formula (XXVII):

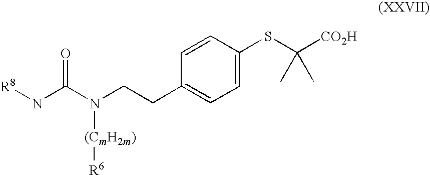

and esters, salts, and physiologically functional derivatives thereof; wherein m is from 0 to 20, R⁶ is selected from the group consisting of hydrogen and

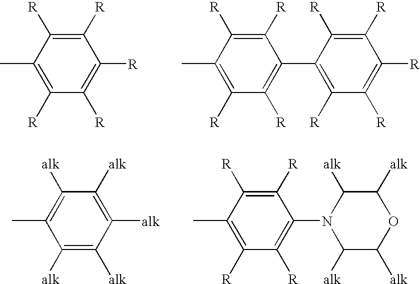

and R⁸ is selected frown the group consisting of:

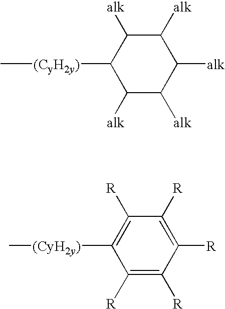

where y is 0, 1, or 2, each alk is independently hydrogen or alkyl group containing 1 to 6 carbon atoms, each R group is independently hydrogen, halogen, cyano, —NO₂, phenyl, straight or branched alkyl or fluoroalkyl containing 1 to 6 carbon atoms and which can contain hetero atoms such as nitrogen, oxygen, or sulfur and which can contain functional groups such as ketone or ester, cycloalkyl containing 3 to 7 carbon atoms, or two R groups bonded to adjacent carbon atoms can, together with the carbon atoms to which they are bonded, form an aliphatic or aromatic ring or multi ring system, and where each depicted ring has no more than 3 alk groups or R groups that are not hydrogen.

In yet other embodiments of the present invention a PPARγ agonist is a compound such as disclosed in U.S. Pat. No. 6,294,580 and/or Liu et al., Biorg. Med. Chem. Lett. 11 (2001) 3111-3113, e.g., having a structure within Formula XXVIII:

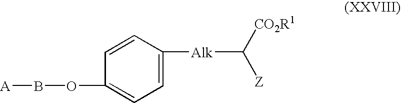

wherein A is selected from the group consisting of: (i) phenyl, wherein said phenyl is optionally substituted by one or more of the following groups; halogen atoms, C₁₋₆alkyl, C₁₋₃ alkoxy, C₁₋₃ fluoroalkoxy, nitrite, or —NR⁷R⁸ where R⁷ and R⁸ are independently hydrogen or C₁₋₃ alkyl; (ii) a 5- or 6-membered heterocyclic group containing at least one heteroatom selected from oxygen, nitrogen and sulfur; and (iii) a fused bicyclic ring

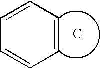

wherein ring C represents a heterocyclic group as defined in point (ii) above, which bicyclic ring is attached to group B via a ring atom of ring C; B is selected from the group consisting of: (iv) C₁₋₆ alkylene; (v) —M C₁₋₆ alkylene or C₁₋₆ alkyleneM C₁₋₆ alkylene, wherein M is O, S, or —NR² wherein R² represents hydrogen or C₁₋₃ alkyl; (vi) a 5- or 6-membered heterocyclic group containing at least one nitrogen heteroatom and optionally at least one further heteroaton selected from oxygen, nitrogen and sulfur and optionally substituted by C₁₋₃ alkyl; and (vii) Het-C₁₋₆ alkylene, wherein Het represents a heterocyclic group as defined in point (vi) above; Alk represents C₁₋₃ alkylene; Het represents hydrogen or C₁₋₃ alkyl; Z is selected from the group consisting of: (viii) nitrogen-containing heterocyclyl or heteroaryl, e.g., N-pyrrolyl, N-piperidinyl, N-piperazinyl, N-morpholinyl, or N-imidazolyl, optionally substituted with 1-4 C₁₋₆ alkyl or halogen substituents; (ix) —(C₁₋₃ alkylene) phenyl, which phenyl is optionally substituted by one or more halogen atoms; and (x) —NR³R⁴, wherein R³ represents hydrogen or C₁₋₃ alkyl, and R⁴ represents C₁₋₆ alkyl, aryl or heteroaryl (e.g., phenyl, pyridinyl, pyrazinyl, pyrimidinyl, pyrrolyl, piperidinyl, piperazinyl, morpholinyl, imidazolyl), optionally substituted by 1-4 C₁₋₆ alkyl, halogen, C₁₋₆ alkoxyl, hydroxyl, nitro, cyano, or amino substituents, or —Y—(C═O)—T—R⁵, —Y—SO₂—R⁵, or —Y—(CH(OH))—T—R⁵, wherein: (a) Y represents a bond, C₁₋₆ alkylene, C₂₋₆ alkenylene, C₄₋₆ cycloalkylene or cycloalkenylene, a heterocyclic group as defined in point (vi) above, or phenyl optionally substituted by one or more C₁₋₃ alkyl groups and/or one or more halogen atoms; (b) T represents a bond, C₁₋₃ alkyleneoxy, —O— or —N(R⁶)—, wherein R⁵ represents hydrogen or C₁₋₃ alkyl; (c) R⁵ represents C₁₋₆ alkyl, C₄₋₆ cycloalkyl or cycloalkenyl, phenyl (optionally substituted by one or more of the following groups; halogen atoms, C₁₋₃ alkyl, C₁₋₃ alkoxy groups, C₁₋₃ alkyleneNR⁹, R¹⁰ (where each R⁹ and R¹⁰ is independently hydrogen, C₁₋₃ alkyl, —SO₂ C₁₋₃ alkyl, or —CO₂ C₁₋₃ alkyl, —SO₂ NH C₁₋₃ alkyl), C₁₋₃ alkyleneCO₂H, C₁₋₃alkyleneCO₂C₁₋₃ alkyl, or —OCH₂C(O)NH₂), a 5- or 6 membered heterocyclic group as defined in point (ii) above, a bicylic fused ring

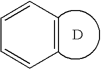

wherein ring D represents a 5- or 6-membered heterocyclic group containing at least one heteroatom selected from oxygen, nitrogen and sulfur and optionally substituted by (═O), which bicyclic ring is attached to T via a ring atom of ring D: or —C₁₋₆ alkyleneMR¹¹M is O, S, or —NR¹² wherein R¹¹ and R¹² are independently hydrogen or C₁₋₃ alkyl, or a tautomeric form thereof, and/or a pharmaceutically acceptable salt or solvate thereof.

One specific group of compounds are those of Formula XI, wherein the dotted line represents no bond, R¹ is methyl, X is O and A is O. Examples of compounds in this group are those compounds where R is phenyl, 2-naphthyl and 3,5 bis(trifluoronethyl)phenyl. Another specific group of compounds are those of Formula XIII, wherein the dotted line represents no bond, R¹ is methyl and A is O. Particularly preferred compounds within this group are compounds where B is CH and R is phenol, p-tolyl, m-tolyl, cyclohexyl, and 2-naphthyl. In alternative embodiments of the present invention, the B is N and R is phenyl.

In still further embodiments, the present invention provides methods for the use of a pharmaceutical composition suitable for administering an effective amount of at least one composition comprising a PPARγ agonist, such as those disclosed herein, in unit dosage form to treat reperfusion related injury associated with reperfusion of ischemic tissue following a cerebrovascular accident. In alternative embodiments, the composition further comprises a pharmaceutically acceptable carrier.

Specific examples of compounds of the present invention are given in the following list: (+)-5[[4-[(3,4-dihydro-6-hydroxy-2,5,7,8-tetramethyl-2H-1-benzopyran-2-yl)methoxy]phenyl]methyl]-2,4thiazolidinedione; (Troglitazone); 5-[4-[2-(5-ethylpyridin-2-yl)ethoxyl]benzyl]thiazolidine-2,4-dione; (pioglitazone); 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; (englitazone); 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione (rosiglitazone); and 5-[4-[2-(N-(benzoxazol-2-yl)-N-methylamino]ethoxy]benzyl]oxazolidine-2,4-di-one.

In yet other embodiments of the present invention, the therapeutic agents comprise compounds having the structure shown in Formula XXIX:

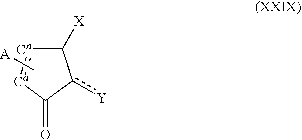

wherein: A is selected from hydrogen or a leaving group at the α- or β-position of the ring, or A is absent when there is a double bond between the Ca and Cn of the ring; X is an alkyl, substituted alkyl, alkenyl, substituted alkenyl, alkynyl, or substituted alkynyl group having in the range of 2 up to 15 carbon atoms; and Y is an alkyl, substituted alkyl, alkenyl, substituted alkenyl, alkynyl, or substituted alkynyl group having in the range of 2 up to 15 carbon atoms. As used herein, the term “leaving group” refers to functional groups which can readily be removed from the precursor compound, for example, by nucleophilic displacement, under E2 elimination conditions, and the like. Examples include, but are limited to, hydroxy groups, alkoxy groups, tosylates, brosylates, halogens, and the like.

The therapeutic agents of the present invention (e.g., the compounds in Formulas I-XXIX and the others described above) are capable of further forming both pharmaceutically acceptable acid addition and/or base salts. All of these forms are within the scope of the present invention and can be administered to the subject to treat acute ischemic stroke and reperfusion related injury following a cerebrovascular accident.

Pharmaceutically acceptable acid addition salts of the present invention include, but are not limited to, salts derived from nontoxic inorganic acids such as hydrochloric, nitric, phosphohoric, sulfuric, hydrobromic, hydriodic, hydrofluoric, phosphorous, and the like, as well as the salts derived forth nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bissulfite, nitrate, phosphate, monoLydrogenphosphate, dihydrogenphosphate, metaphosphate, pyrophosphate, chloride, bromide, iodide, acetate, trifluoracetate, propionate, caprylate, isobutyrate, oxalate, malonate, succinate, suberate, sebacate, fumarate, malcate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like, as well as gluconate, galacturonate, and n-methyl glucamine.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner or as described above. The free base forms differ from their respective salt forms somewhat in certain physical properties such as solubility in polar solvents, but are otherwise equivalent to their respective free base for purposes of the present invention.

Pharmaceutically acceptable base addition salts are formed with metals or amides, such as alkali and alkaline earth metals or organic amines. Examples of metals used as cations include, but are not limited to, sodium, potassium, magnesium, calcium, and the like. Examples of suitable amines include, but are not limited to, N₂—N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid in the conventional manner or as described above. The free acid forms differ from their respective salt forms somewhat in certain physical properties such as solubility in polar solvents, but otherwise the salts are equivalent to their respective free acid for purposes of the present invention.

Certain of the compounds of the present invention can exist in unsolvated forms as well as solvated forms, including, but not limited to, hydrated forms In general, the solvated forms, including hydrated forms, are equivalent to unsolvated forms and are intended to be encompassed within the scope of the present invention. Certain of the compounds of the present invention possess one or more chiral centers and each center may exist in different configurations. The compounds can, therefore, form stereoisomers. Although these are all represented herein by a limited number of molecular formulas, the present invention includes the use of both the individual, isolated isomers and mixtures, including racemates, thereof. Where stereospecific synthesis techniques are employed or optically active compounds are employed as starting materials in the preparation of the compounds, individual isomers may be prepared directly. However, if a mixture of isomers is prepared, the individual isomers may be obtained by conventional resolution techniques, or the mixture may be used as is, with resolution.

Furthermore, the thiazolidene or oxazolidene part of the compounds of Formulas I through XIII can exist in the form of tautomeric isomers, and are intended to be a part of the present invention.

For preparing pharmaceutical compositions from the compounds of the present invention, pharmaceutically acceptable carriers can be in any suitable form (e.g., solids, liquids, gels, etc.). Solid form preparations include, but are not limited to, powders, tablets, pills, capsules, cachets, suppositories, and dispersible granules. A solid carrier can be one or more substances which may also act as diluents, flavoring agents, binders, preservatives, tablet disintegrating agents, or an encapsulating material. The present invention contemplates a variety of techniques for administration of the therapeutic compositions. Suitable routes include, but are not limited to, oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, among others. Indeed, it is not intended that the present invention be limited to any particular administration route.

For injections, the agents of the present invention may be formulated in solutions, such as dimethyl sulfoxide, and/or in physiologically compatible buffers such as Hank's solution, Ringer's solution, or physiological saline buffer. For such transmucosal administration, penetrants appropriate to the barrier to be permeated are used in the formulation. Such penetrants are generally known in the art.

In powders, the carrier is a finely divided solid which is in a mixture with the finely dived active component. In tablets, the active component is mixed with the carrier having the necessary binding properties in suitable proportions, which has been shaped into the size and shape desired.

The powders and tablets preferably contain from five or ten to about seventy percent of the active compounds. Carriers can include, but are not limited to, magnesium carbonate, magnesium stearate, talc, sugar, lactose, pectin, dextrin, starch, gelatin, tragacanth, methylcellulose, sodium carboxymethylcellulose, a low melting wax, cocoa butter and the like, among other embodiments (e.g., solid, gel, and liquid forms). The term “preparation” is intended to also encompass the formation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, in some embodiments of the present invention, a low melting wax, such as a mixture of fatty acid glycerides or cocoa butter; is first melted and the active compound is dispersed homogeneously therein, as by stirring.

The molten homogenous mixture is then poured into convenient sized molds, allowed to cool, and thereby to solidify in a form suitable for administration.

Liquid form preparations include, but are not limited to, solutions, suspensions, and emulsions (e.g., water or water propylene glycol solutions). For parenteral injection, in some embodiments of the present invention, liquid preparations are formulated in solution in aqueous polyethylene glycol solution. Aqueous solutions suitable for oral use can be prepared by dissolving the active component in water and adding suitable colorants, flavors, and stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as paclceted tablets, capsules, and powders in vials or ampoules. Also, the unit dosage form can be a capsule, tablet, cachet, or lozenge itself, or it can be the appropriate number of any of these in packaged form.

The dose, amount, and/or quantity of PPARγ agonist or derivative thereof administered to the subject can depend on the specific PPARγ agonist or derivative thereof selected. For example, troglitazone can be administered to a subject at a dose of about 70 mg/kg, pioglitazone can be administered to a subject at a dose of about 1 mg/kg, rosiglitazone can be administered to a subject at a dose of about 0.1 mg/kg. These doses would translate in doses of about 70 mg pioglitazone, and 7 mg rosiglitazone for an adult human. It will be appreciated that dosages above and below these dosages can be employed and will depend on the potency of the specific compound and the therapeutic regimen employed.

In another aspect of the invention, a thrombolytic agent can be administered to the subject after administering the PPARγ agonist or derivative thereof. The thrombolytic agent can comprise any agent that is effective in helping to dissolve or break up an occuding thrombus. The thrombolytic agent may be selected from those thrombolytic agents, which are known in the art. These include, but are not limited to, streptokinase, urokinase, prourokinase, alteplase, reteplase, anistreplase, and tissue plasminogen activator (t-PA) and biologically active variants thereof. A combination of two or several thrombolytic agents may also be employed.

The thrombolytic agents can be administered using conventional dosage ranges for these agents, for example, a daily dosage used when the agent is administered in thrombolytic therapy as a monotherapy. The range will, of course, vary depending on the thrombolytic agent employed. Examples of dosage ranges are as follows: urokinase-500,000 to 6,250,000 units/patient; streptokinase-140,000 to 2,500,000 units/patient; prourkinase-5,000 to 100,000 units/patient; anistreplase-10 to 100 units/patient; t-PA 0.5 to 2.0 mg/kg body weight.

Thrombolytic therapy for stroke is typically given as an intravenous bolus followed by intravenous infusion or intraarterially as either boluses or short infusions. For intravenous therapy the infusion is completed within an hour; intrarterial treatment may continue for several hours typically up to six hours after the onset of symptoms. Intravenous thrombolytic therapy may comprise administration of up to 10% of the total dose as bolus injection over about 1 to about 5 minutes and the remaining 90% then as a constant infusion during the next hour.

While the entire dose of thrombolytic (0.9 mg/kg for tissue plasminogen activator) is typically administered intravenously, intrarterial therapy is often stopped when reperfusion has been restablished as demonstrated angiographically. The thrombolytic treatment may be supplemented with one or more other active ingredients, e.g., anticoagulants, or surgical methods, such as angioplasty.

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); EM (micromolar); mol (moles); mmol (millimoles); μmolcromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); Sigma (Sigma Chemical Co., St. Louis, Mo.), parts per million (ppm).

### EXAMPLES

### Example 1

### Timing of Reperfusion Determines Neuroprotective Efficacy by Thiazolidinedines

Ischemic injury occurs rapidly after cerebral artery occlusion and this has hampered the development of effective drug therapy for stroke. Development of neuroprotective agents has focused on the “therapeutic window” defined as the interval between ischemic onset and time to drug therapy. The timing of reperfusion relative to drug therapy is becoming more important as reperfusion can be manipulated with thrombolytic agents. Although reperfusion limits ischemic brain injury, it is accompanied by the leukocyte infiltration which exacerbates damage. One class of agents that reduces leukocyte infiltration and ischemic injury when given prior to artery occlusion is the thiazolidinediones (TZDs). We now find that two TZDs, rosiglitazone and pioglitazone, are neuroprotective when given up to three hours after MCAO, but only if reperfusion occurs after treatment. Importantly, delay of reperfusion until after drug administration results in reduced leukocyte infiltration and infarction size, relative to earlier reperfusion times in our model. Additionally, TZD treatment reduces intracellular adhesion molecule (ICAM) expression which mediates leukocyte infiltration. These data have important implications for both mechanisms of neuroprotection and clinical trial design.

Early reperfusion is the foundation of current acute stroke therapy; however, reperfusion can also aggravate ischemic injury through multiple mechanisms. One mechanism implicated in reperfusion injury is inflammatory response mediated by leukocytes which become activated within 30 minutes of ischemic onset. Activated leukocytes adhere to adhesion molecules, including selectins, intracellular cell adhesion molecule (ICAM) and vascular adhesion molecule, on the surface of activated endothelium and this accumulation within the microvasculature can lead to capillary plugging, thereby worsening ischemia. Once adherent, leukocytes can transmigrate through the endothelium and into brain parenchyma. Neutrophil infiltrates are detected as early as one hour after reperfusion and are significant six hours after reperfusion. Monocyte infiltration become significant approximately twenty-four hours after reperfusion. In permanent ischemia leukocyte infiltration occurs at later times and is less robust, mostly occurring at the edge of infarction and not in the ischemic core. Activated leukocytes release proteases, lipid derived mediators and reactive oxygen species (ROS) that can further injure already compromised brain. Neutrophil infiltrates are detected as early as one hour after reperfusion and are significant six hours after reperfusion.

TZDs are insulin sensitizers used to treat type 2 diabetes. Two agents are used clinically, rosiglitazone and pioglitazone. These drugs are agonists of the peroxisome proliferator-activated receptor gamma (PPARγ), a ligand activated transcription factor which regulates fatty acid metabolism, adipocyte development and serum glucose levels. It has become clear that these drugs are also potent anti-inflammatory agents. The mechanism for the anti-inflammatory actions are unclear but may involve direct transcriptional changes as well as modulation of the activity of other transcription factors such as NFκB, AP-1 and STATs. Among the many anti-inflammatory actions of these drugs, TZDs reduce ICAM expression and leukocyte infiltration in several injury paradigms including cardiac, gut and renal ischemial/reperfusion injury. ICAM mRNA is increased one hour after artery occlusion and neutrophil infiltration is first detected at this time. Importantly, studies of gut ischemia show reduced ICAM-IR 3 hours and 15 minutes after TZD treatment showing that TZD effects on leukocyte infiltration occur rapidly. It is important to note that other adhesion molecules such as vascular adhesion molecule, CD8 and selectins have also been found to be reduced by TZDs and reduction in these molecules may contribute to reduced leukocyte infiltration in TZD treated animals.

Two TZDs, pioglitazone and troglitazone, are effective neuroprotective agents in rodent models of cerebral ischemia. Importantly, reductions in infarct size persist weeks after MCAO and are accompanied by improved neurologic function. Additionally TZD pretreatment reduces the inflammatory infiltrate that follows stroke and the expression of inflammatory mediators known to exacerbate injury. Furthermore, ischemic stroke is associated with a dramatic increase in PPARγ expression and is activated when by PPARγ agonists including TZDs.

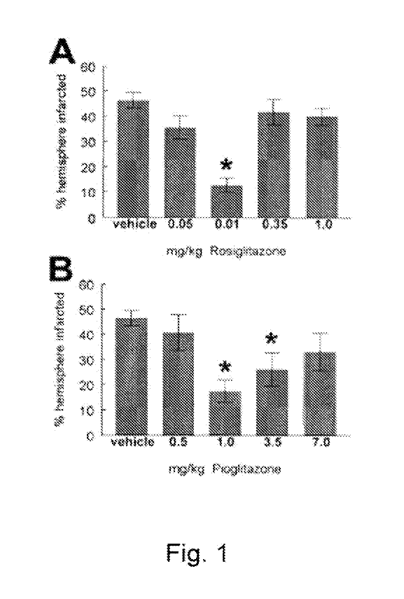

In the current study we examined neuroprotective dose response curves for pioglitazone and rosiglitazone, when injected IP twenty-four hours before and again at the time of infarction. When assayed twenty-four hours after MCAO, the optimal dose for pioglitazone was 1 mg/kg and the optimal dose for rosiglitazone was 0.1 mg/kg (FIG. 1). We used these doses in all further experiments. Higher closes of drugs were not efficacious.

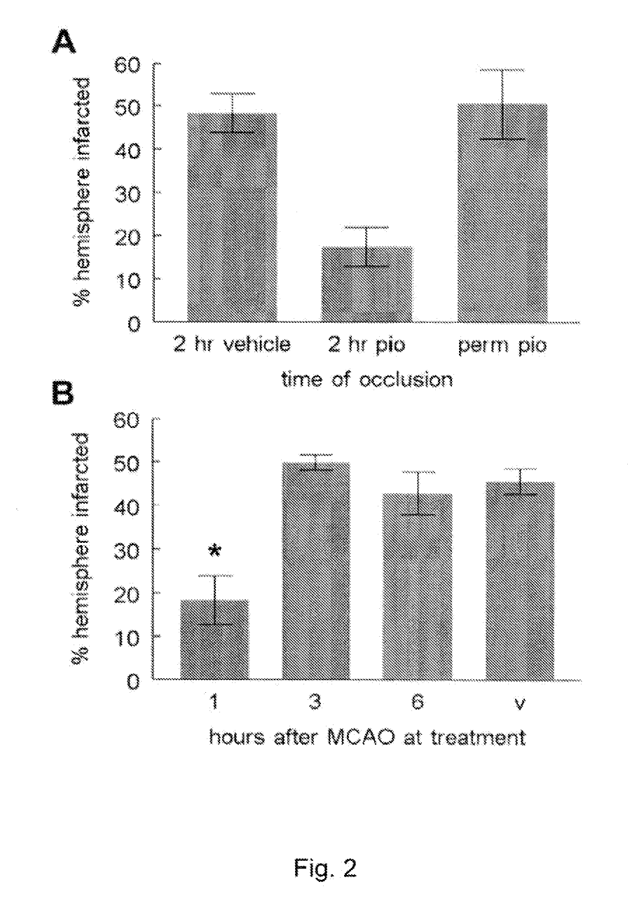

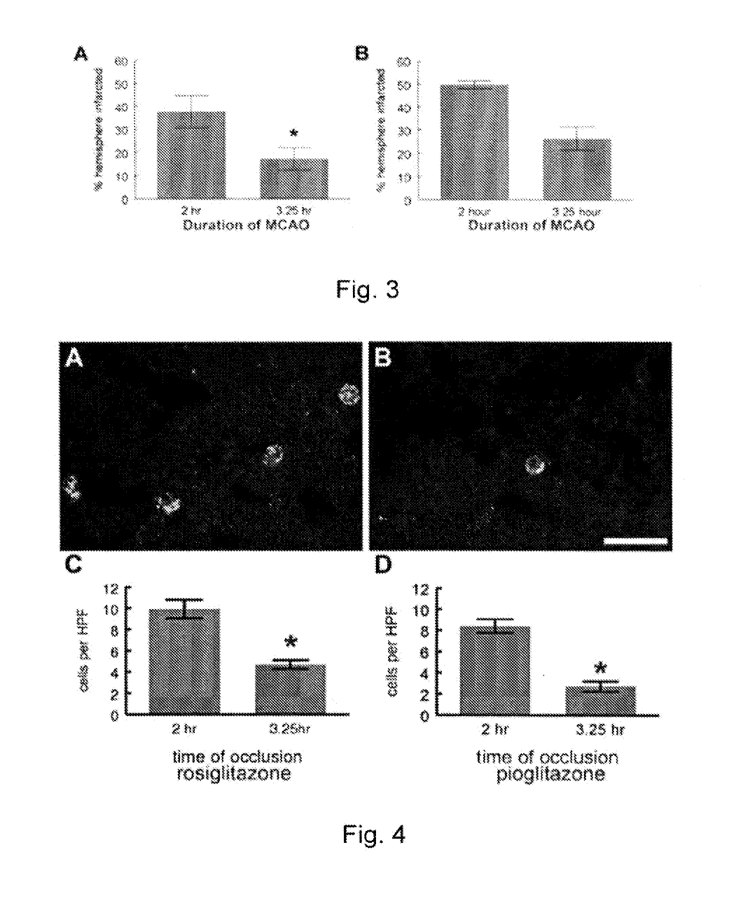

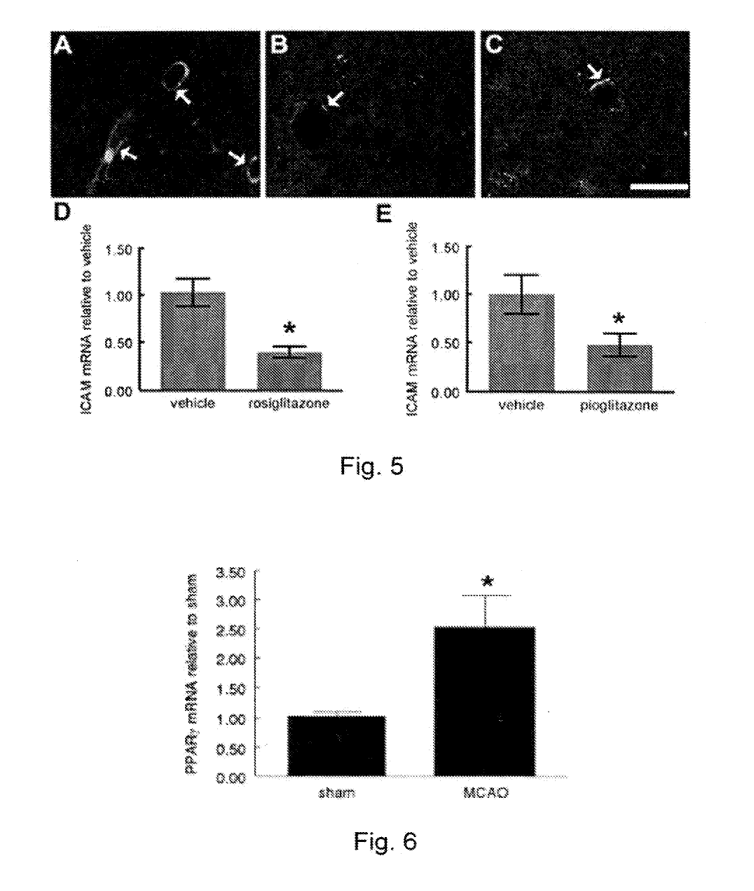

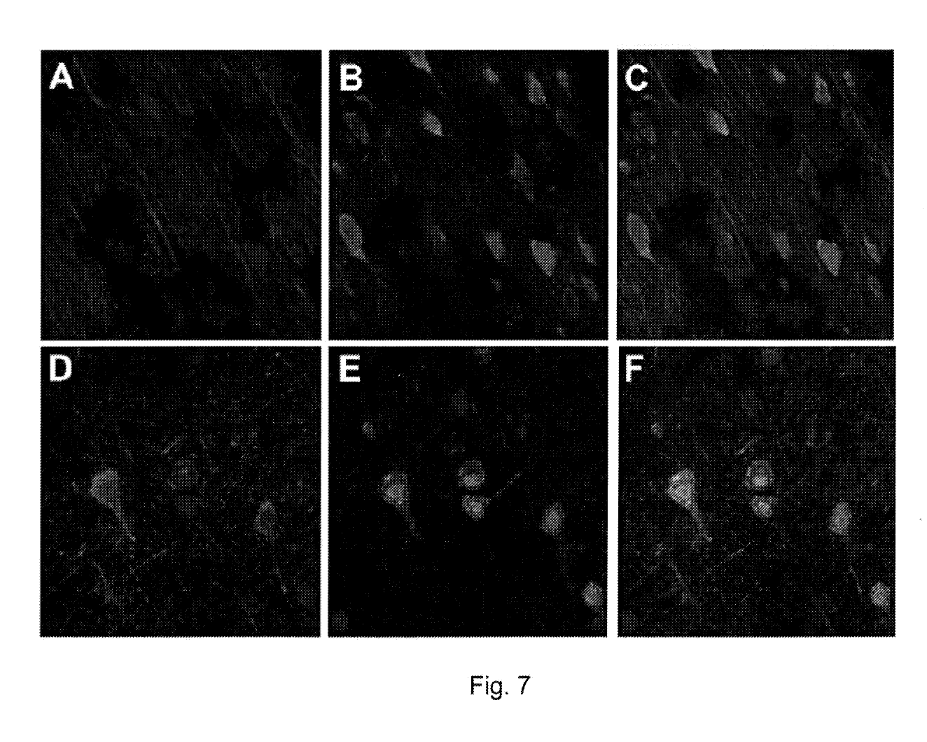

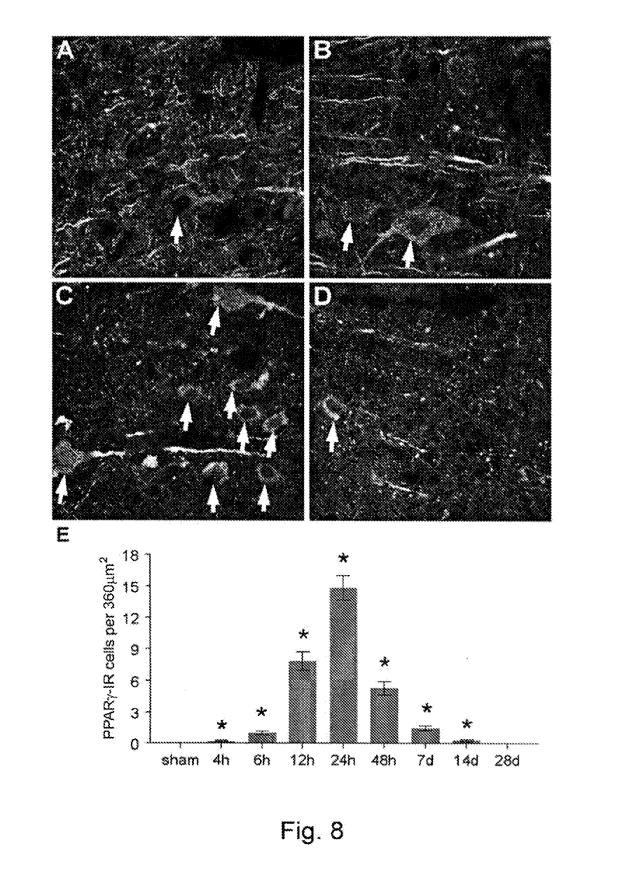

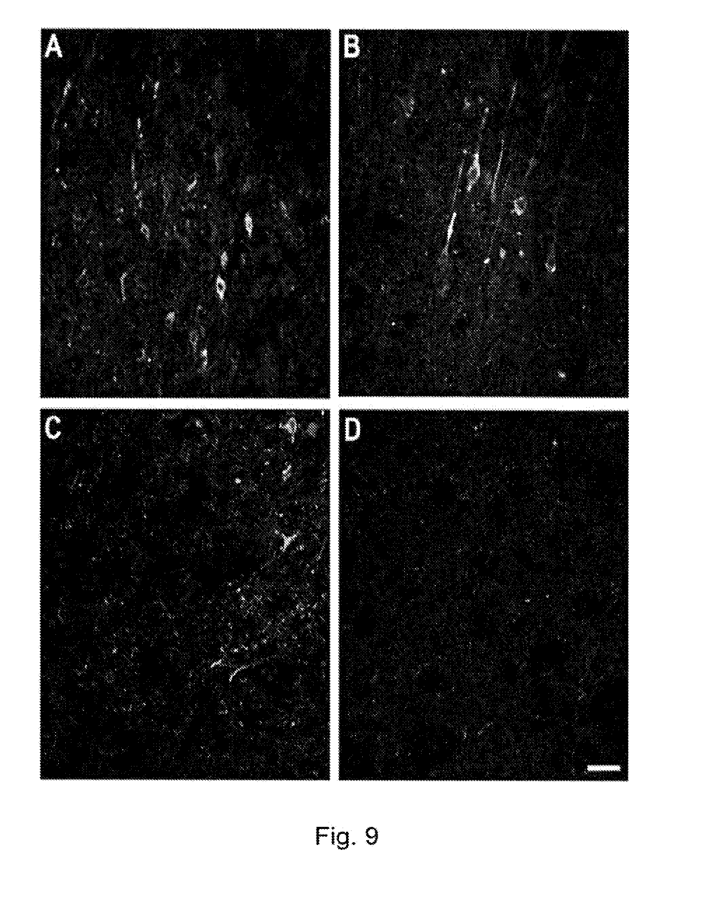

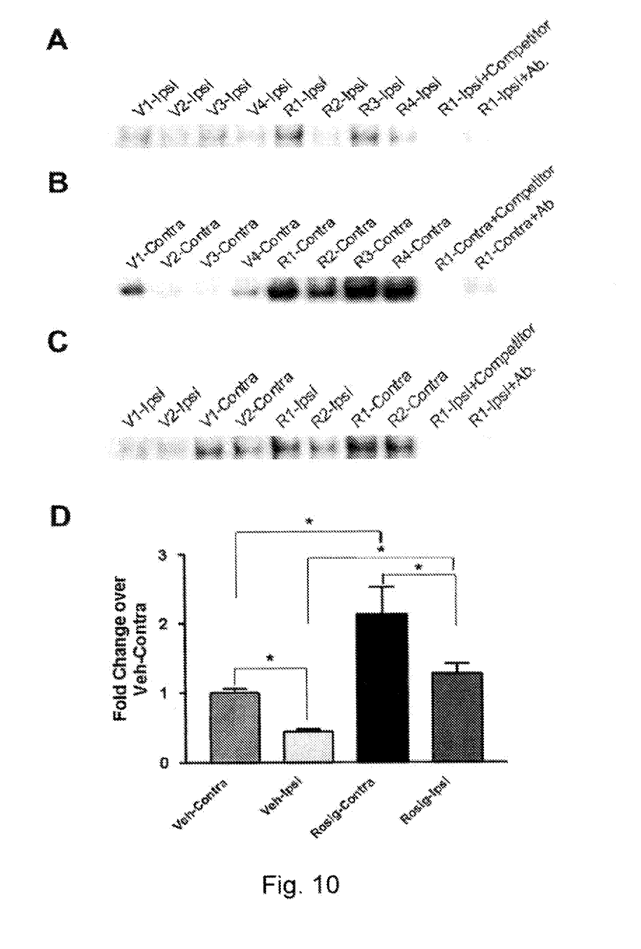

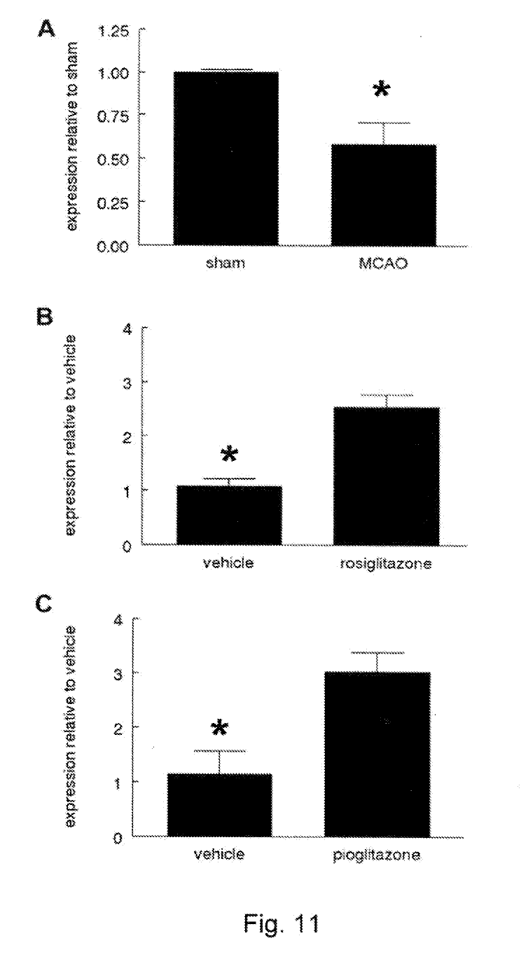

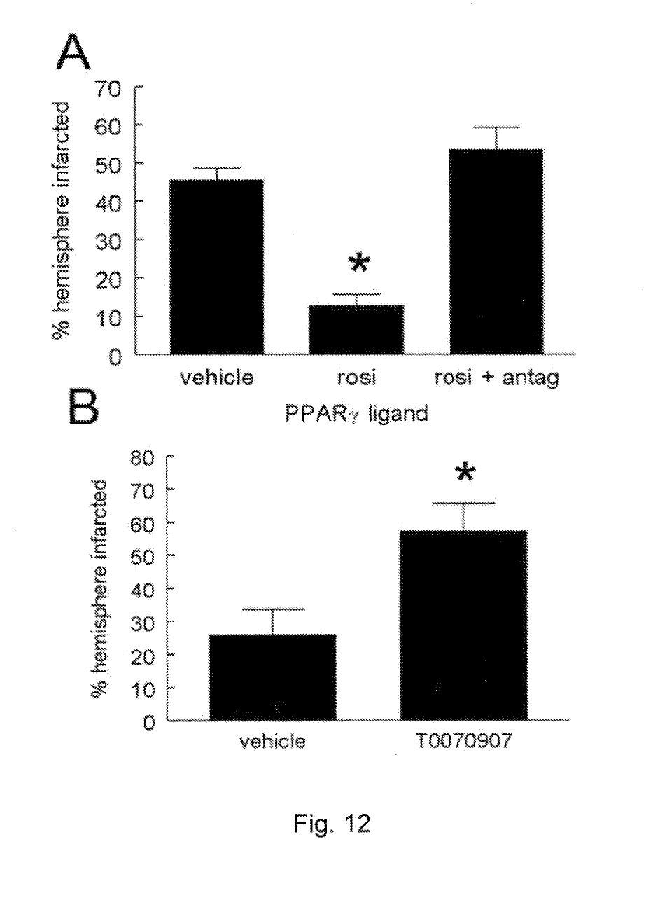

Oral pretreatment with pioglitazone is effective in transient but not permanent ischemia. Using IP injections, we tested the neuroprotective actions of pioglitazone when given twenty-four hours before and again at the time of MCAO in both 2 hour and permanent occlusion models and found significantly reduced infarction size in transient but not permanent MCAO (FIG. 2A). When the effective time window for pioglitazone in a two hour MCAO model was examined, the drug was able to significantly reduce infarction volume when administered one hour after MCAO, but not when given three or six hours after MCAO (FIG. 2B). Taken together these data suggest that TZDs must be given prior to reperfusion to be effective.

We formally tested the hypothesis that TZDs must be given prior to reperfusion to elicit neuroprotective effects by administering either pioglitazone or rosiglitazone to rats three hours after MCAO and altering the time of reperfusion. Half the rats were reperfused at two hours, while the other rats were reperfused at three hours and fifteen minutes. Animals that were reperfused at two hours and treated one hour later had infarction volumes which were similar to animals injected with vehicle; these infarctions encompass the majority of the MCA territory. However, animals that were treated at three hours and reperfused fifteen minutes later had significantly smaller infarctions volumes when assayed twenty-four hours after MCAO (FIG. 3). These infarctions were largely limited to the striatum, in a pattern similar to that seen with TZDs pretreatment.

We examined leukocyte infiltration in TZD treated rats. Myeloperoxidase is present in leukocytes and is easily detected through either enzyme assays or immunohistochemistry. Twenty-four hours after MCAO the vast majority of leukocytes within the brain are neutrophils. Animals reperfused two hours after MCAO and treated with either pioglitazone or rosiglitazone at three hours had significantly increased numbers of myeloperoxidase-IR cells within the ischemic hemisphere relative to the brains of animals which were treated at three hours and reperfused fifteen minutes later (FIG. 4). Myeloperoxidase-IR cells were distributed throughout the ischemic territory in both groups of animals and the degree of reduction in myleoperoxidase-IR was similar with either drug.

TZDs reduce expression of adhesion molecules that medicate leukocyte infiltration including ICAM in several systems. We tested the ability of rosiglitazone and pioglitazone to suppress ICAM expression following cerebral ischemia. Rats were treated with either vehicle, pioglitazone (1.0 mg/kg) or rosiglitazone (0.1 mg/kg) twenty-four hours before and again at the time of occlusion. Rats were sacrificed twenty-four hours and both ICAM-IR by immunohistochemistry and relative ICAM mRNA levels by real time PCR examined. Less ICAM-IR was present in TZD treated rats compared with vehicle treated rats. Furthermore, there was a significant reduction in ICAM mRNA with both TZD treatments relative to the vehicle treatment (FIG. 5). The degree of reduction by pioglitazone and rosiglitazone was similar.

Traditional wisdom is that the most important determinate of final cerebral injury following stroke is the time of occlusion and previous studies have concentrated on the length of time from vessel occlusion when assessing the time window for efficacy. To our knowledge this is the first study to specifically assess whether the time of reperfusion relative to drug administration alters outcome. Reperfusion can occur spontaneously at various times after the onset in human ischemic stroke. Since the advent of thrombolytic therapy for stroke, time of reperfusion is increasingly controlled by the administration of thrombolytics. To date thrombolysis is the only FDA approved therapy for acute ischemic stroke and the practice since its approval has been to administer thrombolytics and then consider if patients might be candidates for neuroprotective trials. These experiments indicate that this approach may not be effective for some therapies, especially those likely to target reperfusion injury.

While there is little data addressing how quickly TZDs reduce the expression of ICAM or other adhesion molecules required for leukocyte infiltration, our data suggest that it is important the process begin before leukocytes have significant access to ischemic endothelium through reperfusion. Earlier reperfusion relative to ICAM suppression provides a longer time period for adhesion and subsequent infiltration. While we do not advocate delaying reperfusion in stroke patients, our data dramatically demonstrates that TZDs must be given prior to reperfusion in order to be effective. It has recently been reported that a non-TZD PPARγ agonist, prostaglandin J2 is beneficial in hemorrhagic stroke. This raises the possibility that TZDs might be safely be given before CT scanning, a strategy that would enable earlier treatment. Our data show that the timing of neuroprotective efficacy is dependent on more than just the time from ischemic onset and that understanding the potential mechanisms of actions may lead to strategies that increase the time window of efficacy for therapy.

### Materials and Methods

### Rats

Male Wistar rats, 250-350 g, were obtained from Charles River (Wilmington, Mass.). Animals were housed and cared for in the Animal Resource Center and allowed free access to food and water before and after surgery. All procedures were approved by the Institutional Animal Care and Use Committee of Case Western Reserve University.

### Middle Cerebral Artery Occlusion

MCAO was achieved in halothane anesthetized rats using the suture method. A 4.0 nylon suture with a flame rounded tip was advanced through the internal carotid artery to occlude the MCA while cerebral blood flow was monitored using laser doppler (Perimed AB, Jarfalla, Sweden). At the time of reperfusion rats were again anesthetized and the suture removed with cerebral blood flow monitoring to ensure reperfusion. Arterial blood pressure, arterial blood gases and temperature were all monitored and controlled during the length of the procedure.

### Drug Treatment

Animals were treated with either pioglitazone (Takeda Pharmaceuticals North America, Lincolnshire, Ill.) or rosiglitazone (GlaxoSmitheKline Pharmaceuticals, London, England) dissolved in 0.1 ml of DMSO and injected interperitoneally. Optimal neuroprotection for pioglitazone (0.5, 1, 3.5 and 7 mg/kg) and rosiglitazone (0.05, 0.1, 0.35, 1 mg/kg) were tested by injecting the drugs 24 hours before and again at the time of 2 hour MCAO. Rats were euthanized 24 hours after MCAO and infarct volumes calculated to determine doses which provided maximal neuroprotection. The optimal doses were found to be 1 mg/kg for pioglitazone and 0.1 mg/kg for rosiglitazone and were used in the subsequent experiments. Control animals were injected with 0.1 ml DMSO.

### Infarct Volume Determination

Rats were euthanized by exposure to 5% halothane until breathing ceased. They then underwent intracardiac perfusion first with PBS and then with 4% paraformaldehyde. Brains were isolated and immersion fixed in 4% paraformaldehyde overnight. Tissue was cryoprotected in 30% sucrose in PBS. Twenty micron frozen sections were collected every 1000 mm beginning at the level of the forceps minor corpus callosum and extending through the hippocampus. Sections were stained with Thionin and the area of infarction measured using Bioquant computer software (R&M Biometrics, Nashville, Tenn.) by subtracting the uninfarcted tissue in the ischemic hemisphere from the volume of the contralateral hemisphere. This method corrects for post-ischemic edema.

### Immunohistochemisiry

Animals were euthanized and brains prepared in the same manner described for infarct volume determination. Frozen sections of the brains were cut coronally at ten microns. Sections were collected every 1000 mm beginning at the level of the forceps minor corpus callosum and extending through the hippocampus. Ten micron cryostat sections were labeled using either mouse anti-ICAM (Sertec, UK) or rabbit antimyeloperoxidase (Dacko) antibodies in 5% rat serum. Primary antibodies were visualized using either goat anti-mouse conjugated to Oregon Green or goat anti-rabbit conjugated to Oregon-Green (Molecular Probes, Eugene, Oreg.) in 5% rat serum. Adjacent sections were incubated without primary antisera to control for non-specific binding of the secondary antiserum. Sections were viewed using a fluorescent microscope and images acquired and stored using Spot Advanced imaging software (Spot Image Corporation, Chantilly, Va.). Some sections were also imaged using confocal microscopy.

### QuantUlcation of Myeloperoxidase-IR Cells

Myeloperoxidase-IR cells were quantified from cryostat sections through the maximal area of infarction labeled with rabbit anti-myeloperoxidase antibody followed by anti-rabbit antibodies conjugated to Oregon-green. Digital photographs of high powered fields covering a representative section were analyzed in Adobe Photoshop by overlaying a grid and counting all myeloperoxidase-IR cells. The average number of myeloperoxidase-IR cells per high powered field (360 μm2) was calculated and plotted using data from at least three animals.

### Real Time PCR

Rat brains were quickly removed immediately following euthanasia, via halothane inhalation. A coronal slice, approximately 3 mm thick, through the area of infarction was cut and homogenized in Trizol (Gibco BRL, Rockville, Md.). This slice contains both penumbra and infarction core as well as uninfarcted tissue. The remaining tissue was placed in TTC (Sigma, St. Louis, Mo.) in order to confirm infarction. RNA isolation and cDNA synthesis, and PCR amplifications were performed as previously described3. Amplification was performed using an I-Cycler detection system (BioRad Laboratories, Hercules, Calif.) with SYBR Green I. ICAM primer (Integrated DNA Technology Inc. Coralville, Iowa) sequences were as follows: sense (5′GAG TCT CCC AGC ACC AGC AT 3′) (SEQ ID NO: 1), anti sense (5′ ATT TAG GCA TGG TGG TTG ACA TT 3′) (SEQ ID NO: 2). Beta actin primer (β-actin; Integrated DNA Technology Inc. Coralville, Iowa) sequences were: sense (5′ AGA GGG AAA TCG TGC GTG AC 3′) (SEQ ID NO: 3) and anti-sense (5′ CCA TAG TGA TGA CCT GTC CGT 3′) (SEQ ID NO: 4). Each PCR amplification was performed in triplicate wells, using the following conditions: 3 minutes at 95° C., 30 seconds at 95° C. and 30 seconds at 60° C. through 40 cycles, followed by 2 cycles of 30 seconds at 72° C. and 1 minute at 60° C. Following amplification melt curve analysis was performed to ensure primer dimmers were not present. Threshold cycle (Ct) values were calculated by determining the point at which emitted fluorescence exceeded the baseline plus ten times baseline standard deviation. Relative expression of the mRNA for ICAM was expressed as the ratio of the mRNA for the housekeeping gene β-actin.

### Example 2

We examined PPARγ expression and activation in adult rat brain following stroke and explore its role in ischemic injury.

### Materials and Methods

### Animals

Male Wistar rats, 250-350 g, were obtained from Charles River (Wilmington, Mass.). Animals were housed and cared for in the Animal Resource Center and allowed free access to food and water. All procedures were reviewed and approved by the Institutional Animal Care and Use Committee (IACUC) at Case Western Reserve University.

### Drug Treatment

Animals treated with PPARγ agonists or antagonists were treated 24 hours before middle cerebral artery occlusion (MCAO) and again at the time of MCAO. Either pioglitazone (0.1 mg/kg; Takeda Pharmaceuticals North America, Lincolnshire, Ill.), rosiglitazone (0.1 mg/kg; GlaxoSmithKline Pharmaceuticals, London, England) and/or the PPARγ antagonist, T0070907 (1.5 mg/kg; Caymen Chemical; Ann Arbor, Mich.), were dissolved in dimethylsulfoxide (DMSO). Our previous experiments have shown that there is no effect of DMSO on infarction size in rats injected with 0.1 ml DMSO/250 g body weight compared with untreated rats. In addition, a similar degree of neuroprotection was found using rosiglitazone in a dilute DMSO solution. The final concentrations we injected were pioglitazone: 0.25 mg/ml, rosiglitazone: 0.25 mg/ml and T0070907: 3.75 mg/ml. These concentrations were chosen so that equal volumes of DMSO would be injected in all animals (0.1 ml per 250 g of body weight). The concentration of T0070907 is three times the concentration at which 50% inhibition of [3H] rosiglitazone binding occurs. Agents were injected intraperitoneally with the animal under anesthesia (1.5% halothane in 30% oxygen and 68.5% nitrous oxide).

### Middle Cerebral Artery Occlusion

Male Wistar rats, 250-350 g, were obtained from Charles River (Wilmington, Mass.). These rats have been extensively used in similar experiments and show good reproducibility with respect to infarct size. Animals were housed and cared for in the Animal Resource Center and allowed free access to food and water before and after surgery. Following anesthesia induction with 3% halothane, rats were ventilated with 1.5% halothane in 30% oxygen and 68.5% nitrous oxide delivered via facemask. Normothermia was maintained by a rectal telethermometer coupled to a 125W infrared light source until animals recovered from anesthesia. The tail artery was exposed and cannulated to measure mean arterial blood pressure and to obtain blood for blood gas measurements during surgery. Animals in which normal temperature, blood pressure and gases could not be maintained were excluded from the study. MCAO was performed as previously described. Briefly, the bifurcation of the common carotid artery was exposed. The external carotid artery was isolated and a suture placed 0.5 cm from the bifurcation. Following electrocoagulation distal to the suture the external carotid was cut between the suture and the electrocoagulated segment. A suture was applied to the common carotid artery such that it could be removed later. A loop of suture was then placed under the internal carotid artery and the artery elevated to transiently occlude blood flow. A small nick was made in the wall of the external carotid artery. Through this nick a 22 mm length of 4.0 Ethicon suture, its tip rounded by heating near a flame, was gently threaded from the external to the internal carotid artery lumen to a distance of 18 mm from the bifurcation of the carotid. The thread was held in place with a suture around the stump of the external carotid artery and the suture around the internal carotid artery released. The catheter was removed from the tail artery and both incisions were closed. The rats were then allowed to recover from anesthesia. At the time of reperfusion, which in most cases was two hours after the thread was placed, the animals were induced with 3% halothane and anesthesia maintained with 1.5% halothane in 30% oxygen and 68.5% nitrous oxide delivered by face mask. The neck incision was reopened and the 4.0 Ethicon suture removed. The suture that held it in place was tightened and the suture around the common carotid artery was removed. After confirmation of reperfusion by visual inspection, the incision was closed and the animal allowed to recover.

### Cerebral Blood Perfusion Monitoring

Male Wistar rats were anesthetized with halothane. Cerebral blood perfusion was monitored during surgery and reperfusion including the time of occlusion and the time of reperfusion. A small craniotomy was made midway between the corner of the eye and the external auditory canal. A laser Doppler probe (Perimed AB, Järfälla, Sweden) was placed such that its beam passed through the craniotomy and cerebral blood perfusion measured. Once the baseline measurement was obtained, MCAO was performed. Cerebral blood perfusion was monitored at the time of MCAO and at the time of reperfusion. There were no significant differences in cerebral blood flow reductions during MCAO for any of the treatment groups. Following completion of MCAO, animals were allowed to recover and returned to their cages.

### Animal Euthanasia and Tissue Preseivation

At the time of sacrifice the animal was placed in an anesthesia chamber and exposed to 5% halothane, 30% oxygen, 65% nitrous oxide until it ceased breathing. The rat was the, processed for either histologic and immunocytochemical analysis or decapitated and the brain immediately dissected for mRNA isolation. Animals used for immunohistochemistry underwent intracardiac perfusion; the thoracic cavity was opened, a blunt needle placed through the left ventricle into the aorta and the right atrium cut. Animals were perfused through the heart, first with 0.1 M phosphate buffer and then with 4% paraformaldehyde (pH 7.4). The brain was removed from the rat and immersion fixed in 4% paraformaldehyde for an additional hour. Tissue was then transferred to 30% sucrose in phosphate buffer in order to cyroprotect it. Brains were mounted in OCT sectioning medium and frozen sections cut at either twenty microns (for infarct volume determination) or ten microns (for immunocytochemistry). Animals used in real time PCR experiments were decapitated after euthanasia and the brain quickly removed. A coronal slice, approximately 3 mm thick, through the area of infarction was cut and placed in Trizol. In rats where infarction was not readily visualized at dissection, a small slice of adjacent tissue was placed in 2,3,5-triphenyl-tetrazolium chloride (Sigma, St. Louis, Mo.) in order to confirm infarction; infarct volume was not calculated in animals used for real time PCR as tissue was homogenized.

### Real Time PCR

A 2 mm thick coronal slice of tissue through the area of maximal infarction was quickly homogenized in Trizol (Gibco BRL, Rockville, Md.). This slice contains both penumbra and infarction core as well as uninfarcted tissue. RNA was isolated using Life Technologies Protocol (Rockville, Md.), and cDNA synthesized from 1.5 μg of total RNA. PCR amplifications were performed in the BioRad I-Cycler detection system (BioRad Laboratories, Hercules, Calif.) in a total volume of 25 μl containing 100 mM KCl, 40 mM Tris-HCl pH 8.4, 0.4 mM of each dNTP, iTaq DNA polymerase 50 units/ml, 6 mM MgCl2, SYBR Green I 20 nM fluorescein, and stabilizers. Each reaction also contained either 100 nM PPARγ, 300 nM lipoprotein lipase primers or 300 nM β-actin primers (table 1; Integrated DNA Technology Inc. Coralville, Iowa). Each PCR amplification was performed in triplicate wells, using the following conditions: 3 minutes at 95° C., 30 seconds at 95° C. and 30 seconds at 60° C. through 40 cycles, followed by 2 cycles of 30 seconds at 72° C. and 1 minute at 60° C. The final two cycles are included in order to perform melt curve analysis to assure that there were no primer dimers. Threshold cycle (Ct) values were calculated by determining the point at which emitted fluorescence exceeded the baseline plus ten times baseline standard deviation.

### Immunohistochemistry

Cryostat sections were rinsed in phosphate buffered saline (PBS), preincubated in dilution buffer (0.5 M sodium chloride, 0.01 M phosphate buffer, 3% bovine serum albumin, 0.1% sodium azide, 0.3% Triton-X 100), and then incubated in dilution buffer containing goat serum and primary antibodies (Table 2) in dilution buffer with 5% rat serum overnight. The sections were rinsed in PBS and then incubated with secondary antibodies in dilution buffer with 5% goat serum for two hours before being rinsed in PBS again. Sections were mounted in glycerol:PBS (1:1) containing 1% N-propyl gallate. In some cases immunocytochemical signals were amplified using biotinylated secondary antisera. When this was done, sections were incubated with primary antisera as described above, rinsed in PBS and then incubated with either goat anti-mouse or goat anti-rabbit biotin conjugated antibodies in dilution buffer with 5% goat serum for two hours. Sections were rinsed in PBS and incubated with strepavidin conjugated to Cy-3 in PBS for one hour. Following this, slides were rinsed in PBS and coverslipped. With each experiment adjacent sections were incubated without primary antisera to control for non-specific binding of the secondary antiserum. Sections were viewed using a fluorescent microscope and images acquired and stored using Spot Advanced imaging software (Spot Image Corporation, Chantilly, Va.). Some sections were also imaged using confocal microscopy.

### Counting of PPARγ-IR Cells

Since PPARγ-IR was heterogeneously distributed within sections of the brain, we counted all the PPARγ-IR cells in the one coronal section through the center of the infarction. These sections contain striatum, the ischemic core, and the cortex. One section from each of at least 3 animals was randomly selected and digital images obtained of every field in the hemisphere ipsilateral to the infarction. Images were then overlaid with a grid in Adobe Photoshop and all PPARγ-IR cells counted. The average number of PPARγ-IR cells per high powered field was calculated and plotted.

### Electrophorectic Mobility Shift Assay

Rats were treated with either vehicle (DMSO) or rosiglitazone (0.1 mg/kg in DMSO) twenty-four hours before and again at the time of 2 hour MCAO. Eight hours after the onset of MCAO the animals were euthanized by halothane inhalation and the brains quickly removed. Blocks of tissue 4 mm thick encompassing the rostral caudal extension of the infarct and the same area on the contralateral hemisphere were dissected free and snap frozen in liquid nitrogen. Nuclear extraction was performed and protein concentrations were determined using the Bradford method.

Ten micrograms of nuclear extract protein was incubated with 32P-labeled double stranded oligonucleotide (40 fmol) containing the PPAR responsive element sequences in rat acyl CoA oxidase promoter (5′-AGTCCTTCCCGAACGTGACCTTTGTCCTGGTCCCCTTTTGCTC-3′) (SEQ ID NO: 5). For competition assays, 100 fold radioinert (cold) competitor oligonucleotides were included in the reaction mixture. Interference assays with antibody to PPARγ (2 μg/reaction; SC-7273x, Santa Cruz Biotechnology,) were used to determine specificity of PPARγ-PPRE binding.

Nucleoprotein-oligonucleotide complexes were resolved electrophoretically on a 7% non-denaturing polyacrylamide gel. The gel was autoradiographed and optical density was assessed using Kodak Analysis (EDAS) 290 system.

### Infarct Volume Calculation

Twenty micron frozen sections of paraformaldehyde fixed brains were collected every 1000 μm beginning at the level of the forceps minor corpus callosum and extending through the hippocampus. The slides were stained with Thionin. The sections were then trans-illuminated and the image transmitted to a computer screen. The area of infarction was measured using Bioquant computer software. In order to minimize the effects of edema on infarction size calculations, the area of uninfarcted tissue in the ischemic hemisphere was measured and subtracted from the area of the contralateral hemisphere. The area of infarction from each slide was summed and presented as a percentage of the volume of the uninfarcted hemisphere. This method of infarct volume calculation has been previously described.

### Results

### PPARγ Expression

PPARγ expression was investigated using real time PCR and immunohistochemistry. Samples from real time PCR were run in triplicate on two separate occasions. There was good reproducibility between triplicates and between different experiments. Brain tissue from rats that underwent a sham operation and were sacrificed 24 hours later contained a low level of PPARγ mRNA as evidenced by high cycle thresholds. However, in brain tissue from rats subjected to 2 hour MCAO there was a 2.5 fold increase in PPARγ mRNA levels in the ipsilateral hemisphere. This represents a statistically significant difference (FIG. 6; student's t-test; p<0.05). PPARγ protein expression was examined using immunohistochemistry in brains from rats 24 hours after either a sham operation or two hour MCAO followed by reperfusion. PPARγ-IR fibers but not immunoreactive cell bodies were present within the cortex of sham operated rats and rats, which did not undergo any procedure. Following MCAO, there was a dramatic increase in PPARγ-IR. This increase occurred initially, within the cortex of infarcted brain, but by twenty-four hours was most evident at the edges of the infarction in the ischemic penumbra. We were unable to detect nuclear PPARγ-IR using z-sectioning with confocal microscopy at all time points examined. Morphologically, these cells appeared to be neurons. This was verified by double labeling with mouse antibodies directed against the neuronal marker, NeuN, and rabbit antisera directed against PPARγ (FIG. 7). We were unable to identify non-neuronal PPARγ-IR cells at any time examined. At later time points auto-fluorescence of macrophages interfered with double labeling of microglia/macrophages. Labeling with the non-florescent chromogens Vector Blue and Nova Red did not reveal any PPARγ-IR cells with non-neuronal morphology (data not shown).

### Up-Regulation of PPAR Occurs Quickly and is Long-Lasting

Ischemia rapidly induces PPARγ expression with elevated levels of receptor-IR observed as early as four hours after MCAO. The number of PPARγ-IR fibers and cell bodies progressively increased until twenty-four hours following MCAO. By three days the number of PPARγ-IR cell bodies and fibers dramatically diminish. Rare PPARγ-IR cells with neuronal morphology are detected up to fourteen days after MCAO but by twenty-eight days after MCAO PPARγ-IR was equivalent to that seen in untreated or sham operated animals (FIG. 8). Quantification of these immunohistochemical data show that the number of PPARγ-1R cells per high power field was significantly increased in ischemic rats relative to sham operated animals at four, six, twelve and twenty-four hours as well as at two, three, seven, and fourteen days after MCAO (student's t test p<0.05). The density of PPARγ-IR cell bodies was not significantly different in animals sacrificed one hour after MCAO or twenty-eight days after MCAO (student's t test p>0.05).

PPARγ-IR was primarily found in the cortex (FIG. 9). Immunoreactive cell bodies were found in the anterior cerebral artery territory adjacent to the ischemic middle cerebral artery territory and throughout the cortex supplied by the middle cerebral artery. In general, PPARγ-IR was less evident in the temporal lobe, which tends to suffer severe ischemia, at twenty-four hours. The striatum, which represents the ischemic core, does not possess PPARγ-IR.

Ischemia Reduces PPRE Binding while a PPARγ Agonist Increases PPRE Binding

Rats treated with vehicle (DMSO) or rosiglitazone (0.1 mg/kg in DMSO), a PPARγ agonist, twenty-four hours before and again at the time of two hour MCAO were evaluated by electrophoretic mobility shift assay for PPARγ DNA binding. Despite increased PPARγ expression in neurons, we found overall reduced DNA binding in the ischemic hemisphere. Rosiglitazone treatment, on the other hand increased DNA binding in both the ischemic and the non-ischemic hemispheres. Densitometry was used to quantitate the results and showed that the ischemic hemisphere of vehicle treated rats had only 44% of the binding seen in the contralateral hemisphere. In rosiglitazone treated rats there was a 59% decrease in the ischemic hemisphere relative to the contralateral hemisphere (FIG. 10). In both sets of animals the reductions were significantly different (p<0.05; students t-test). Addition of the agonist, rosiglitazone, significantly increased DNA binding (p<0.05; students t-test). In the contralateral hemisphere there was 213% increase, while in the ischemic hemisphere the increase was 289% relative to vehicle treated rats. It should be noted that there were no differences in physiologic parameters including temperature, arterial blood gases, blood pressure or percentage reduction in cerebral blood flow during MCAO between rats treated with either vehicle or rosiglitazone.

Ischemia Reduces PPARγ Target Gene mRNA while PPARγ Agonists Increase Target Gene mRNA

Lipoprotein lipase (LPL) plays an important role in lipid metabolism; its promoter contains a PPRE and expression is increased by PPARγ activation. LPL is expressed by a variety of neurons. We used real time PCR to monitor mRNA levels of LPL in the affected hemisphere from rats treated with either MCAO sham operation or rats treated with 2 hour MCAO. These data were run in triplicate, on three separate occasions. There was good reproducibility among triplicates, different runs and between the two primer sets. LPL mRNA was significantly decreased after MCAO relative to approximately half that found in sham operated rats (FIG. 11A p<0.05). Brains from animals undergoing 2 hour MCAO had half the concentration of LPL mRNA of brains from animals undergoing a sham operation. These data are consistent with the observation that ischemia reduces PPARγ binding to the PPRE. Treatment of rats with PPARγ agonists twenty-four hours before and again at the time of 2 hour MCAO resulted in a 254% (rosiglitazone 0.1 mg/kg) and 307% (pioglitazone 1.0 mg/kg) increase in LPL mRNA levels within the ischemic hemisphere (FIG. 11B). These data were statistically significant (p>0.05). Changes in LPL expression roughly minor the magnitude of changes in PPARγ DNA binding seen by electrophoretic mobility shift assay. Changes in LPL expression can not be explained by changes in physiologic parameters with agonist treatment since physiologic parameters including temperature, arterial blood gases, hypertension and percentage reduction in cerebral blood flow did not differ in vehicle and agonist treated rats.

Infarction Size is Increased Following Treatment with the PPARγ Antagonist, T000907

Although PPARγ agonists are protective in transient ischemia models, it has been unclear if these effects are PPARγ dependent or independent. We utilized T0070907, a PPARγ antagonist which blocks agonist induced recruitment of co-activators to PPARγ and promotes recruitment of co-repressor proteins thereby suppressing DNA binding to determine if PPARγ played a role in PPARγ agonist mediated neuroprotection. T0070907 is a selective PPARγ antagonist and has a greater than 800 fold preference for PPARγ over PPARα and PPARδ. In competition with the PPARα and PPARδ co-ligand [3H] GW2433, T0070907 has an apparent Ki of 0.85 mM to PPARα and 1.8 mM to PPARδ. Rats were injected with the PPARγ agonist rosiglitazone, T0070907 and rosiglitazone dissolved in DMSO or DMSO twenty-four hours before and again at the time of two hour MCAO. Injection of agonists had no significant effect on cerebral blood flow, arterial blood pressure, arterial blood gases or temperature during the time of surgery. We have previously shown that animals injected with pioglitazone have no significant change in cerebral blood flow during two hour continuous monitoring. Animals were sacrificed twenty-four hours after MCAO and infarct volumes calculated. Rosiglitazone treated rats had significantly smaller infarct on volumes than vehicle injected rats. In contrast, infarction volumes in rats treated with both rosiglitazone and the PPARγ antagonist were not significantly different from those seen in vehicle treated rats (FIG. 12A; student's t-test; p<0.05). In addition, we treated separate animals with either vehicle or T0070907 without agonist to determine if PPARγ was protective in the absence of exogenous ligand. These animals were treated twenty-four hours before and at the time of 90 minute MCAO. The shorter infarction time was chosen so that the infarction volume of vehicle treated animals would be small and we would be able to detect an increase in infarction size if antagonism of PPARγ were detrimental. As expected these vehicle injected animals had infarction sizes which were smaller than that seen with 2 hour occlusions. T0070907 treated rats, however, had infarctions that encompassed essentially the entire MCA distribution and were significantly larger than the volumes found in vehicle treated rats (FIG. 12B; student's t-test p<0.05). These data suggest that the neuroprotective actions of rosiglitazone are PPARγ dependent and furthermore that PPARγ activation, most likely by endogenous ligands, is protective following cerebral ischemia in untreated rats.

### Discussion

PPARγ agonists reduce infarction size and improve neurologic function in cerebral ischemia. Both PPARγ dependent and independent actions of PPARγ agonists have been described. We wanted to understand the expression and activation of PPARγ in the brain during cerebral ischemia. We found that PPARγ expression is increased following ischemia. Surprisingly, however, we found that DNA binding of PPARγ is reduced in ischemic brain and that transcription of a PPARγ dependent gene is similarly reduced. This reduction is overcome by treatment with a PPARγ agonist. We also show that rosiglitazone, a PPARγ agonist reduces infarction size. This reduction is blocked by treatment with the PPARγ antagonist, T0070907. Importantly, we find that the antagonist also increases infarction size in the presence or absence of exogenous agonist, suggesting that endogenous ligands also limit ischemic injury.

Several examples have demonstrated that PPARγ is regulated by tissue injury. PPARγ is dramatically increased in macrophages during peritonitis and in an arthritic model. PPARγ-IR is also increased in smooth muscle cells following balloon injury and in spontaneously hypertensive rats while there are modest increases in PPARγ-IR in endothelial cells, synovial cells and fibroblasts in tissue from patients with rheumatoid arthritis. Furthermore, PPARγ-IR is present in the CNS of experimental autoimmune encephalitis mice, but not control mice. Interestingly, animal models of arthritis, experimental autoimmune encephalitis and cerebral ischemia have been found to benefit from PPARγ ligands and this benefit is accompanied by reduced proinflammatory gene expression.

We observe a dramatic increase in cytoplasmic PPARγ-IR. Cytoplasmic PPARγ-IR has been reported in a variety of tissues including 3T3-L1 preadipocytes, macrophages, vascular smooth muscle cells and synovial cells. In adult rat neurons it was found that PPARα and -β-IR were exclusively nuclear while, PPARγ-IR was also cytoplasmic. PPARγ has been reported to translocate to the nucleus and bind DNA following treatment with PPARγ agonists. While we have not visualized nuclear PPARγ-IR, we have been able to demonstrate DNA binding and modulation of PPARγ target gene expression in response to TZDs indicating that nuclear PPARγ is present at levels below the level of detection of our immunohistochemical protocol.

The significance of cytosolic PPARγ is unclear. Another steroid nuclear transcription factor, the estrogen receptor, is found in dendrites and axons where it binds estrogens and regulates neurotransmitter and endothelial nitric oxide synthase release. Estrogen receptors functionally interfere with the degradation of IκBα and NFκB nuclear translocation without influencing transcription. Since these actions do not involve DNA binding they are referred to as “nongenomic”. Non-genomic actions of PPARγ have also been described: PPARγ can transrepress other transcription factors by binding coactivator proteins or PPARγ can directly bind transcription factors preventing DNA binding.

While PPARγ expression as measured by real time PCR and immunohistochemistry increased following ischemia, DNA binding and transcription of a target gene was reduced. These data show that PPARγ transcriptional activity does not mirror PPARγ levels and suggest that regulation occurs at another level. Corepressors including nuclear receptor corepressor and silencing mediator of retinoid and thyroid hormone receptors interact with other corepressors such as Sin3 and histone deacetylases which inhibit transcription by altering chromatin structure. Recently, these co-repressors have been shown to decrease PPARγ transcriptional activity in 3T3-L1 cells. The addition of a TZD, pioglitazone, results in dissociation of the PPARγ-corepressor complex and transcriptional activation. Interestingly, interaction with co-repressors reduces PPARγ degradation. It is quite possible that the observed increase in PPARγ-IR is a consequence of reduced activation following ischemia.

We tested whether neuroprotection by TZDs is mediated by PPARγ activation using a recently developed PPARγ antagonist, T0070907. This antagonist blocks agonist induced recruitment of coactivator molecules and promotes the recruitment of nuclear receptor corepressor to PPARγ. T0070907 is highly specific for PPARγ having a greater than 800 fold preference for PPARγ over PPARα and PPARγ. We found that the PPARγ agonist, rosiglitazone reduced infarction volume by approximately 75%, when administered twenty-four hours before and at the time of 2 hour MCAO; this protection was completely lost when T0070907 was given along with rosiglitazone (FIG. 12A). These data in combination with the demonstrated activation of PPARγ in response to TZD provide compelling evidence that the protective actions of TZDs are mediated by PPARγ.

We were also able to show that treatment with the antagonist increased infarction size in the absence of exogenous agonist suggesting that even the low level of PPARγ activation that occurs during ischemia is protective. PPARγ has a large ligand binding site which can bind a variety of structurally diverse ligands. Several fatty acids and eicosanoids bind and activate PPARγ at micromolar concentrations and these concentrations are consistent with the levels found in serum. Conversion of 9-hydroxy-10,12-octadecadienoic acid and 13-hyroxy-9,11-octadecadienoic acid by 15-lipoxygenase produces additional products which bind PPARγ at micromolar levels. 15-deoxy-D12,14-prostaglandin J2 is a weak PPARγ ligand and binds at concentrations of 2-5 μM. Additionally, an oxidized alkyl phospholipids, hexadecyl azelaoyl phosphatidyicholine binds PPARγ with a Kd of approximately 40 nM. Nitrolinoleic acid, a derivative of linolic acid, has also recently been reported to bind PPARγ with affinities similar to rosiglitazone (Ki>1 000 nM). While our data support the concept of endogenous ligands and indicate that they are protective in ischemia, it should be noted that an alternative explanation for enhanced injury by PPARγ antagonism is that the antagonist has PPARγ independent toxicity.

There is a substantial body of evidence to support a protective role for PPARγ in neurons. Troglitazone reduces cell death in cultured cerebellar granule neurons following glutamate exposure and suppresses low-potassium-induced apoptosis in the same cells. Neuroprotection is present even when troglitazone is added two and one-half hours after the end of glutamate exposure suggesting that troglitazone interferes with downstream consequences of glutamate activation. Injection of three PPARγ agonists, troglitazone, ibuprofen and 15-deoxy-D12,14-prostaglandin J2 reduces cell death in rat cerebellum exposed to bacterial lipopolysaccharide and interferon-γ. Pioglitazone also attenuates dopaminergic cell death in a Parkinson's disease model. Finally, PPARγ activation in hippocampal neurons is protective against amyloid-β induced neurodegeneration. Importantly, PPARγ agonists demonstrate protective actions in the setting of ischemia. Treatment with a variety of PPARγ agonists reduces infarction size following myocardial ischemia and is also beneficial in pulmonary and renal ischemia.

The mechanisms by which PPARγ agonists and antagonists influence infarction size remain unclear. Our antagonist data indicate that PPARγ is likely involved in ligand mediated neuroprotection as well as endogenous mechanisms of protection. PPARγ ligands may act by binding DNA and regulating transcription or they may act independently of DNA binding. Changes in PPARγ DNA binding may well alter transcriptional regulation, while changes in PPARγ expression may have a different impact on non-genomic PPARγ actions. It is also not clear if the observed changes in PPARγ expression are related to the protective effects of PPARγ agonists or if these changes are perhaps a compensatory response to altered PPARγ activation. While peak changes in expression occur at a time when infarct size is largely developed, significant changes in PPARγ activation and expression are evident within a few hours of ischemia onset and during the time when the inflammatory response to stroke is developing. The kinetics of intraperitoneal PPARγ ligands is unknown, however, the half life of oral TZDs is approximately 4-9 hours and so it is likely that these drugs are present during the time that PPARγ activation and expression is altered. Since FDA approval PPARγ agonists are widely used in the treatment of type 2 diabetes, and are currently being investigated for the treatment of other conditions. An understanding of the mechanisms and consequences of PPARγ activation and expression in ischemic disease is, therefore, of particular importance.

TABLE 1
PCR Primers

| | | | Amplicon |
|---|---|---|---|
| | Forward | Reverse | Size |
| β-actin | 5′-AGAGGGAAATCGTCGTGAC-3′ | 5′-CCATAGTGATGACCTGTCCGT-3′ | 135 |
| | (SEQ ID NO: 6) | (SEQ ID NO: 7) | |
| PPARγ | 5′-CACAATGCCATCAGGTTTGG-3′ | 5-CCATAGTGATGACCTGTCCGT-3′ | 81 |
| | (SEQ ID NO: 8) | (SEQ ID NO: 9) | |
| | Primer Set 1: | Primer Set 1: | |
| LPL | 5′ AAA GTC AGA GCC AA GAGA | 5′ CCA GAA AAG TGA ATC TTG ACT | 98 |
| | AGC A-3′ | TGG T-3′ | |
| | (SEQ ID NO: 10) | (SEQ ID NO: 11) | |
| | Primer Set 2: | Primer set 2: | |
| LPL | 5′-TGG GTG TGT AAG GAA GCT | 5′ CAG ATA GCC ACA ACA GCG TTT | 60 |
| | TGT AAA-3′ | C-3′ | |
| | (SEQ ID NO: 12) | (SEQ ID NO: 13) | |

TABLE 2
Antibodies used Immunocytochemistry

| Antibody | Company | Concentration |
|---|---|---|
| | Primary Antisera | |
| Mouse Anti Neun | Chemicon International Inc. | 1/500 |
| Rabbit Anti PPARγ (H-100) | Santa Cruz Biotechnology | 1/50 |
| | Secondary Antisera | |
| Oregon Green 488 anti mouse 1gG | Molecular Probes | 1/100 |
| Oregon Green 488 anti-rabbit 1gG | Molecular Probes | 1/100 |
| Donkey Anti Mouse Biotin | Jackson Immuno Research Laboratories, Inc. | 1/200 |
| Goat Anti Rabbit 1gG | Chemicon International Inc. | 1/200 |
| | Avidin Conjugated | |
| Cy-3conjuaged*Streptavidin | Jackson Immuno Research Laboratories, Inc. | 1/50 |

Having described the invention, the following is claimed:

1. A method of treating cerebrovascular accident in a subject, the method comprising: administering a therapeutically effective amount of at least one PPARγ agonist or a derivative thereof to the subject after ischemia sufficient to a cause the cerebrovascular accident and prior to reperfusion.

2. The method of claim 1, the PPARγ agonist or derivative thereof being administered to the subject in an amount effective to suppress to ICAM expression in the subject.

3. The method of claim 1, the PPARγ agonist or derivative thereof being administered at an amount effective to suppress leukocyte infiltration to ischemic tissue of the subject.

4. The method of claim 1, the PPARγ agonist or derivative thereof being administered at an amount effective to mitigate reperfusion related ischemic injury.

5. The method of claim 1, the PPARγ agonist or derivative thereof being administered intravenously to the subject.

6. The method of claim 1, further comprising administering a thrombolytic agent after administering the PPARγ agonist or derivative thereof.

7. The method of claim 1, the PPARγ agonist or a derivative thereof comprising a compound of Formula I or pharmaceutically acceptable salt of a compound of Formula I, wherein Formula I is: 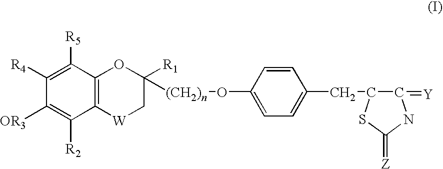
wherein R₁ and R₂ are the same or different, and each represents a hydrogen atom or a C₁-C₅ alkyl group;
R₃ represents a hydrogen atom, a C₁-C₆ aliphatic acyl group, an alicyclic acyl group, an aromatic acyl group, a heterocyclic acyl group, an araliphatic acyl group, a (C₁-C₆ alkoxy)carbonyl group, or an aralkyloxycarbonyl group;
R₄ and R₅ are the same or different, and each represents a hydrogen atom, a C₁-C₅ alkyl group or a C₁-C₅ alkoxy group, or R₄ and R₅ together represent a C₁-C₅ alkylenedioxy group;
n is 1, 2, or 3;
W represents the CH₂, CO, or CHOR₆ group in which R₆ represents any one of the atoms or groups defined for R₃; and
Y and Z are the same or different and each represents an oxygen atom or an imino (—NH) group; and pharmaceutically acceptable salts thereof.

8. The method of claim 1, the PPARγ agonist or a derivative thereof comprising a compound of Formula II or pharmaceutically acceptable salt of a compound of Formula II, wherein Formula II is: 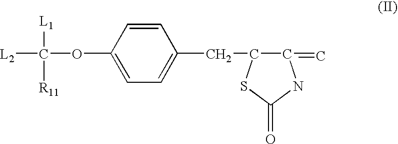
wherein R₁₁ is a substituted or unsubstituted alkyl, alkoxy, cycloalkyl, phenylalkyl, phenyl, aromatic acyl group, a 5- or 6 membered heterocyclic group including 1 or 2 heteroatoms selected from the group consisting of nitrogen, oxygen, and sulfur, or a group of the formula indicated in: 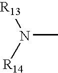
wherein R₁₃ and R₁₄ are the same or different and each is lower alkyl; and wherein L¹ and L² are the same or different and each is hydrogen or lower alkyl or L¹ and L² are combined to form an alkylene group.

9. The method of claim 1, the PPARγ agonist or a derivative thereof comprising a compound of Formula III or pharmaceutically acceptable salt of a compound of Formula III, wherein Formula III is: 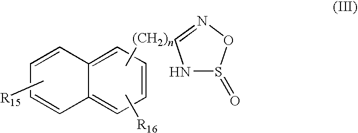
wherein R₁₅ and R₁₆ are independently hydrogen, lower alkyl containing 1 to 6 carbon atoms, alkoxy containing 1 to 6 carbon atoms, halogen, ethyl, nitrite, methylthio, trifluoromethyl, vinyl, nitro, or halogen substituted benzyloxy; and n is 0 to 4.

10. The method of claim 1, the PPARγ agonist or a derivative thereof comprising a compound of Formula IV or pharmaceutically acceptable salt of a compound of Formula IV, wherein Formula IV is: 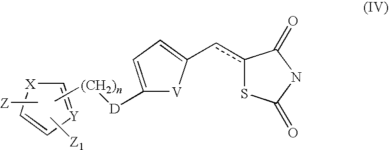
wherein the dotted line represents a bond or no bond; V is HCH—, —NCH—, —CH═N—, or S;
D is CH₂, CHOH, CO, C═NOR₁₇, or CH═CH; X is S, SO, NR₁₈, —CH═N, or —N═CH;
Y is CH or N;
Z is hydrogen, (C₁-C₇)alkyl, (C₁-C₇)cycloalkyl, phenyl, naphthyl, pyridyl, furyl, thienyl, or phenyl mono- or di-substituted with the same or different groups which are (C₁-C₃)alkyl, trifluoromethyl, (C₁-C₃)alkoxy, fluoro, chloro, or bromo
Z₁ is hydrogen or (C₁-C₃)alkyl;
R₁₇ and R₁₈ are each independently hydrogen or methyl; and n is 1, 2, or 3.

11. The method of claim 1, the PPARγ agonist or a derivative thereof comprising a compound of Formula V or pharmaceutically acceptable salt of a compound of Formula V, wherein Formula V is: 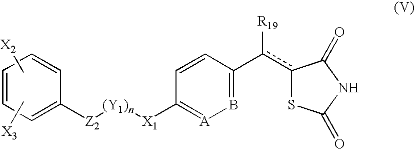
wherein the dotted line represents a bond or no bond;
A and B are each independently CH or N with the proviso that when A or B is N the other is CH; X is S, SO, SO₂, CH₂, CHOH, or CO;
n is 0 or 1;
Y₁ is CHR₂₀ or R₂₁, with the proviso that when n is 1 and Y₁ is NR₂₁, X₁ is SO₂ or CO; Z₂ is CHR₂₂, CH₂CH₂, cyclic C₂H₂O, CH═CH, OCH₂, SCH₂, SOCH₂, or SO₂CH₂;
R₁₉, R₂₀, R₂₁, and R₂₂ are each independently hydrogen or methyl; and
X₂ and X₃ are each independently hydrogen, methyl, trifluoromethyl, phenyl, benzyl, hydroxy, methoxy, phenoxy, benzyloxy, bromo, chloro, or fluoro.

12. The method of claim 1, the PPARγ agonist or a derivative thereof comprising a compound of Formula II or pharmaceutically acceptable salt of a compound of Formula VI, wherein Formula VI is: 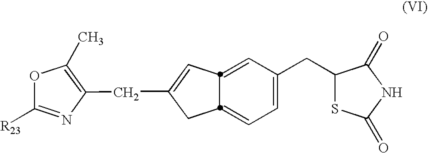
wherein R₂₃ is alkyl of 1 to 6 carbon atoms, cycloalkyl of 3 to 7 carbon atoms, phenyl or mono- or all-substituted phenyl wherein the substituents are independently alkyl of 1 to 6 carbon atoms, alkoxy of 1 to 3 carbon atoms, halogen, or trifluoromethyl.

13. The method of claim 1, the PPARγ agonist or a derivative thereof comprising a compound of Formula VII or pharmaceutically acceptable salt of a compound of Formula VII, wherein Formula VII is: 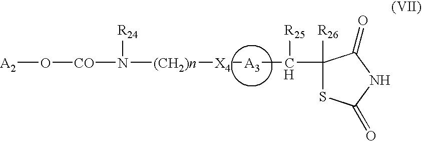
wherein A² represents an alkyl group, a substituted or unsubstituted aryl group, or an aralkyl group wherein the alkylene or the aryl moiety is substituted or unsubstituted;
A³ represents a benzene ring having in total up to 3 optional substituents;
R₂₄ represents a hydrogen atom, an alkyl group, an acyl group, an aralkyl group wherein the alkyl or the aryl moiety is substituted or unsubstituted, or a substituted or unsubstituted aryl group; or A² together with R₂₄ represents substituted or unsubstituted C₂₋₃ polymethylene group;
R₂₅ and R₂₆ each represent hydrogen, or R₂₅ and R₂₆ together represent a bond; X₄ represents O or S; and
n represents an integer in the range from 2 to 6.

14. The method of claim 1, the PPARγ agonist or a derivative thereof comprising a compound of Formula VIII or pharmaceutically acceptable salt of a compound of Formula VIII, wherein Formula VIII is: 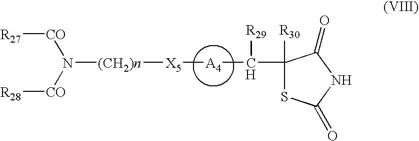
wherein: R₂₇ and R₂₈ each independently represent an alkyl group, a substituted or unsubstituted aryl group, or an aralkyl group being substituted or unsubstituted in the aryl or alkyl moiety;
or R₂₇ together with R₂₈ represents a linking group, the linking group consisting or an optionally substituted methylene group or an O or S atom; R₂₉ and R₃₀ each represent hydrogen, or R₂₉ and R₃₀ together represent a bond;
A₄ represents a benzene ring having in total up to 3 optional substituents;
X₅ represents O or S; and
n represents an integer in the range of 2 to 6.

15. The method of claim 1, the PPARγ agonist or a derivative thereof comprising a compound of Formula IX or pharmaceutically acceptable salt of a compound of Formula IX, wherein Formula IX is: 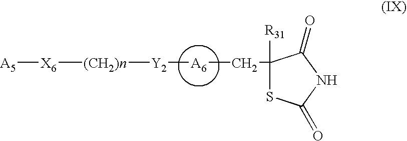
wherein: A₅ represents a substituted or unsubstituted aromatic heterocyclyl group; A₆ represents a benzene ring having in total up to 5 substituents;
X₆ represents O, S, or NR₃₂ wherein R₃₂ represents a hydrogen atom, an alkyl group, an acyl group, an aralkyl group, wherein the aryl moiety may be substituted or unsubstituted, or a substituted or unsubstituted aryl group;
Y₂ represents O or S;
R₃₁ represents an alkyl, aralkyl, or aryl group; and n represents an integer in the range from 2 to 6.

16. The method of claim 1, the PPARγ agonist or a derivative thereof comprising a compound of Formula X or pharmaceutically acceptable salt of a compound of Formula X, wherein Formula X is: 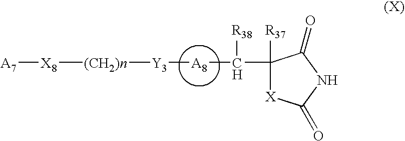
wherein: A₇ represents a substituted or unsubstituted aryl group;
A₈ represents a benzene ring having in total up to 5 substituents;
X₈ represents O, S, or NR₉, wherein R₃₉ represents a hydrogen atom, an alkyl group, an acyl group, an aralkyl group, wherein the aryl moiety may be substituted or unsubstituted, or a substituted or unsubstituted aryl group;
Y₃ represents O or S;
R₃₇ represents hydrogen;
R₃₈ represents hydrogen or an alkyl, aralkyl, or aryl group or R₃₇ together with R₃₈ represents a bond; and
n represents an integer in the range from 2 to 6.

17. The method of claim 1, the PPARγ agonist or a derivative thereof comprising a compound of Formula II or pharmaceutically acceptable salt of a compound of Formula XI, wherein Formula XI is: 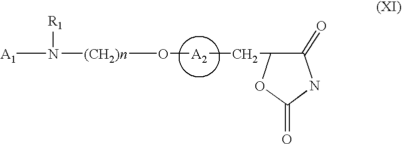
wherein A₁ represents a substituted or unsubstituted aromatic heterocyclyl group;
R¹ represents a hydrogen atom, an alkyl group, an acyl group, an aralkyl group, wherein the aryl moiety may be substituted or unsubstituted, or a substituted or unsubstituted aryl group;
A² represents a benzene ring having in total 1 up to 5 substituents; and n represents an integer in the range of from to 6.

18. The method of claim 1, the PPARγ agonist or a derivative thereof comprising a compound of Formula XII or Formula XIII or pharmaceutically acceptable salt of a compound of Formula XII or Formula XIII, wherein Formula XII and Formula XIII are: 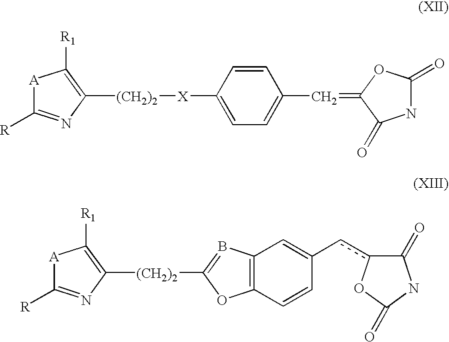
wherein the dotted line represents a bond or no bond;
R is cycloalkyl of three to seven carbon atoms, naphthyl, thienyl, furyl, phenyl, or substituted phenyl wherein the substituent is alkyl of one to three carbon atoms, alkoxy of one to three carbon atoms, trifluoromethyl, chloro, fluoro, or bis(trifluoromethyl);
R₁ is alkyl of one to three carbon atoms;
X is O or C═O;
A is O or S; and
B is N or CH.

19. The method of claim 1, the PPARγ agonist or a derivative thereof comprising at least one compound or a pharmaceutically salt thereof selected from the group consisting of:
(+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4-dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

20. A method of treating reperfusion related injury in a subject following a cerebrovascular accident, the method comprising administering a therapeutically effective amount of at least one PPARγ agonist or a derivative thereof to the subject following onset of the cerebrovascular accident and prior to reperfusion.

21. The method of claim 20, the PPARγ agonist or derivative thereof being administered intravenously to the subject.

22. The method of claim 20, further comprising administering a thrombolytic agent after administering the PPARγ agonist or derivative thereof.

23. The method of claim 20, the PPARγ agonist or the derivative thereof comprising a thiazolidinedione or a derivative thereof.

24. The method of claim 20, the PPARγ agonist or a derivative thereof comprising at least one compound or a pharmaceutically salt thereof selected from the group consisting of: (+)-5[[4-[(3,4-dihydro-6-hydroxy-2,5,7,8-tetramethyl-2H-1-benzopyran-2-yl)ₘ ethoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.
